(19) United States
(12) Patent Application Publication
(10) Pub. No.: US 2026/0311878 A1
(43) Pub. Date: Oct. 8, 2026

(54) METHOD AND ELECTRONIC DEVICE FOR PROVIDING INFORMATION FOR DETERMINING CLINICALLY SIGNIFICANT PROSTATE CANCER BASED ON MULTIMODAL BIOMARKER ADAPTIVE SPATIAL ATTENTION

(71) Applicant: UIF (University Industry Foundation), Yonsei University, Seoul (KR)

(72) Inventors: Jongjin YOON, Seoul (KR); Jongsoo LEE, Seoul (KR); Hyunho HAN, Seoul (KR); Woong Kyu HAN, Seoul (KR); Won Sik HAM, Seoul (KR); Young Taik OH, Seoul (KR); Dae Chul JUNG, Seoul (KR)

(73) Assignee: UIF (University Industry Foundation), Yonsei University, Seoul (KR)

(21) Appl. No.: 19/577,784

(22) Filed: Mar. 25, 2026

(30) Foreign Application Priority Data
Apr. 2, 2025 (KR) 10-2025-0042768
Jan. 15, 2026 (KR) 10-2026-0008280

(51) Int. Cl. G16H 50/20; G06T 7/00; G06T 7/11; G06T 7/62; G16H 10/40; G16H 10/60; G16H 50/30

(52) U.S. Cl. CPC G16H 50/20; G06T 7/0012; G06T 7/11; G06T 7/62; G16H 10/40; G16H 10/60; G16H 50/30; G06T 2207/10081; G06T 2207/10088; G06T 2207/20004; G06T 2207/20084; G06T 2207/30081; G06T 2207/30096

(57) ABSTRACT

The present disclosure relates to a method and an electronic device for providing information related to prostate diseases. More specifically, The present disclosure relates to a technology that utilizes three-dimensional voxel data and an imaging feature map generated from prostate medical image data acquired from magnetic resonance imaging (MRI) or computed tomography (CT), together with non-image clinical data including a serum prostate-specific antigen (PSA) level and past diagnostic history. Based on a Biomarker-Adaptive Spatial Attention Strategy, attention weights of a multimodal cross-attention module are dynamically modulated to detect suspected cancer lesions and determine clinical significance (Clinically Significant Prostate Cancer, CSPC). The technology provides integrated diagnostic information including a Tumor Burden Ratio and a Corrected Prostate-Specific Antigen Density (Corrected PSAD) together with the detection and determination results.

### CROSS-REFERENCE TO RELATED APPLICATION

This application claims priority to and the benefit of Korean Patent Application No. 10-2025-0042768, filed on Apr. 2, 2025, and Korean Patent Application No. 10-2026-0008280, filed on Jan. 15, 2026, the disclosure of which is incorporated herein by reference in its entirety.

### BACKGROUND

### 1. Field of the Invention

The present disclosure relates to a method and an electronic device for providing information related to prostate diseases. More specifically, The present disclosure relates to a technology that utilizes three-dimensional voxel data and an imaging feature map generated from prostate medical image data acquired from magnetic resonance imaging (MRI) or computed tomography (CT), together with non-image clinical data including a serum prostate-specific antigen (PSA) level and past diagnostic history. Based on a Biomarker-Adaptive Spatial Attention Strategy, attention weights of a multimodal cross-attention module are dynamically modulated to detect suspected cancer lesions and determine clinical significance (Clinically Significant Prostate Cancer, CSPC). The technology provides integrated diagnostic information including a Tumor Burden Ratio and a Corrected Prostate-Specific Antigen Density (Corrected PSAD) together with the detection and determination results.

### 2. Discussion of Related Art

The present disclosure is proposed in view of a background in which a gap between image-based diagnosis and clinical indicator-based evaluation has continuously been pointed out in a field where early detection of prostate diseases and prognosis assessment are directly related to the establishment of treatment strategies for patients. Prostate diseases may exhibit differences in lesion location, size, distribution, and progression patterns among patients, and such characteristics may also change over time even in the same patient. Accordingly, diagnostic decisions relying on a single criterion often encounter limitations.

Medical imaging diagnosis of the prostate has improved in accuracy with advancements in imaging devices such as magnetic resonance imaging (MRI) and computed tomography (CT). However, variability depending on the experience and expertise of interpreters may still occur, and due to complex signal characteristics of multi-parameter imaging, there are cases in which lesion boundaries are unclear or it is difficult to distinguish benign changes from malignant changes. In particular, in cases involving small lesions, diffuse lesions, or accompanying inflammatory responses of surrounding tissues, it is not easy to consistently determine clinical significance based solely on imaging.

In addition, it is known that the prostate anatomically includes a peripheral zone and a transition zone having different histological characteristics and occurrence patterns, and the frequency at which lesions are observed and typical imaging findings may vary depending on the zone. However, in actual diagnostic processes, such zonal characteristics are often insufficiently reflected and may be processed according to the same diagnostic criteria, or the zone classification itself may depend on image quality and segmentation accuracy, making it difficult to obtain reliable results.

Meanwhile, biomarkers such as serum prostate-specific antigen (PSA) levels are widely used in the evaluation of prostate diseases. Here, a biomarker refers to a measurable indicator derived from biological samples that indirectly represents a biological condition associated with the presence, progression, treatment response, or risk of a disease, and may include numerical or categorical information obtained from test results of blood, urine, tissue, or the like.

However, biomarkers may not be specific only to a particular disease, and an increase in the measured value may be influenced by various factors such as inflammation, benign prostatic hyperplasia, or medical procedures, rather than being limited to cancer. Therefore, diagnostic decisions based only on a biomarker value at a single time point may lead to false positives or false negatives, and may increase uncertainty in clinical decision-making when imaging results are inconsistent with biomarker-based interpretations.

To address such limitations, attempts have been increasing to consider non-image clinical data such as prostate-specific antigen trends at multiple time points, biopsy history, and treatment history together. However, different forms of data are often managed in a fragmented manner and rely on the intuitive judgment of clinicians. In particular, procedures for systematically combining the temporal order and event context of past patient histories with imaging interpretation results have not been standardized, making it difficult to implement reproducible combined decision-making in actual clinical environments.

Recently, techniques using deep learning-based artificial intelligence models have been proposed to automatically detect or segment lesions in prostate images. However, many models are trained mainly based on features derived from imaging signals, and therefore may not sufficiently reflect clinical contexts such as biomarker variation patterns, zone-specific occurrence characteristics, or risk factors such as recurrence after treatment. In addition, it is often difficult to explain the basis for results presented by such models, which may hinder medical professionals from trusting and adopting the results.

Furthermore, quantitative indicators such as tumor burden, represented by a ratio of lesion volume to total prostate volume, and corrected prostate-specific antigen density calculated using a non-lesion prostate volume obtained by excluding lesions, may quantitatively represent patient conditions. However, when such indicators are calculated in a manner consistently linked with image-based detection results, reliability of the indicators may decrease due to problems such as segmentation errors, boundary uncertainty, and integration methods for multiple lesions. Consequently, even when quantitative indicators are provided, it may be difficult to ensure a level of stability and interpretability suitable for clinical use.

In addition, information required in clinical practice is not limited to simple scores or probability values, but should be provided in the form of explainable integrated information including lesion location, zone assignment, temporal change patterns, and the basis for risk assessment. However, existing approaches often present image-based results and clinical data-based results separately, or the integration process is opaque, making it difficult for users to understand the meaning and basis of the results. Accordingly, there is a need for a method and apparatus capable of providing explainable integrated diagnostic information by combining MRI-or CT-based prostate imaging information with non-image clinical data and biomarker information while reflecting zonal characteristics and temporal contexts.

### SUMMARY OF THE INVENTION

The present disclosure is intended to solve the above-mentioned problems and has the following objectives.

Various embodiments of The present disclosure provide a method and an apparatus for providing information related to prostate diseases by combining MRI-or CT-based prostate medical image data with non-image clinical data and biomarker information.

Various embodiments of The present disclosure provide a method and an apparatus configured to adjust sensitivity of image analysis on a zone-by-zone basis by reflecting characteristics of a peripheral zone (Peripheral Zone, PZ) and a transition zone (Transition Zone, TZ) of the prostate.

Various embodiments of The present disclosure provide a method and an apparatus configured to generate a Biomarker-Adaptive Spatial Attention Strategy based on non-image clinical data including a serum prostate-specific antigen (PSA) level and past diagnostic history, and to perform multimodal fusion by modulating attention weights assigned to spatial locations within an imaging feature map according to the strategy.

Various embodiments of The present disclosure provide a method and an apparatus configured to introduce a time-responsive weight reflecting a temporal context of diagnosis and treatment included in the non-image clinical data so that regions having a higher likelihood of lesion occurrence, expansion, or progression are emphasized.

Various embodiments of The present disclosure provide a method and an apparatus configured to automatically detect an entire prostate region and one or more suspected cancer lesions based on a modulated imaging feature map, and to calculate a lesion volume and a total prostate volume to provide a Tumor Burden Ratio.

Various embodiments of The present disclosure provide a method and an apparatus configured to calculate a Corrected Prostate-Specific Antigen Density (Corrected PSAD) using a non-lesion prostate volume obtained by excluding a lesion volume from a total prostate volume, thereby reducing uncertainty in PSA-based interpretation that may be distorted by prostate size or non-cancerous factors.

Various embodiments of The present disclosure provide a method and an apparatus configured to provide integrated diagnostic information including at least one of the Tumor Burden Ratio and the Corrected PSAD together with a determination result of whether a suspected cancer lesion corresponds to Clinically Significant Prostate Cancer (CSPC), thereby supporting diagnosis and treatment planning by medical professionals.

Various embodiments of The present disclosure provide a method and an apparatus configured such that the integrated diagnostic information is provided in a form in which image-based evidence and clinical data-based evidence are consistently combined, thereby improving reliability and reproducibility of multimodal prostate disease analysis.

The technical problems to be solved by The present disclosure are not limited to those mentioned above, and other technical problems not mentioned herein will be clearly understood by those skilled in the art from the following description.

According to various embodiments of The present disclosure, there is provided a method performed by an electronic device. The electronic device comprises an input device, an output device, a processor, and a memory. The method comprises: acquiring, by the input device, two-dimensional medical image data related to a prostate of a patient from a magnetic resonance imaging (MRI) device or a computed tomography (CT) device; generating, by the processor, three-dimensional voxel data of the prostate based on the two-dimensional medical image data; acquiring, by the input device, non-image clinical data including a serum prostate-specific antigen (PSA) level of the patient and a past diagnostic history of the patient; applying, by the processor, a feature extraction network to the three-dimensional voxel data to generate an imaging feature map, and generating a zone map identifying a peripheral zone (PZ) and a transition zone (TZ) of the prostate within the three-dimensional voxel data; determining, by the processor, a Biomarker-Adaptive Spatial Attention Strategy based on the non-image clinical data, wherein the strategy includes a zone sensitivity asymmetrically assigned to the peripheral zone (PZ) and the transition zone (TZ) according to the PSA level or the past diagnostic history, and a time-responsive weight according to a likelihood of lesion progression; operating, by the processor, a multimodal cross-attention module according to the determined strategy to dynamically modulate attention weights assigned to spatial locations within the imaging feature map; analyzing, by the processor, the modulated imaging feature map to detect an entire prostate region and one or more suspected cancer lesions, and calculating a total prostate volume and a lesion volume of the suspected cancer lesion; calculating, by the processor, a Tumor Burden Ratio, which is a ratio of the lesion volume to the total prostate volume, and a Corrected Prostate-Specific Antigen Density (Corrected PSAD) obtained by dividing the PSA level by a non-lesion prostate volume obtained by excluding the lesion volume from the total prostate volume; and outputting, by the output device, integrated diagnostic information including the Tumor Burden Ratio and the Corrected PSAD together with a determination result indicating whether the suspected cancer lesion corresponds to Clinically Significant Prostate Cancer (CSPC).

According to various embodiments of The present disclosure, there is provided an electronic device. The electronic device comprises: an input device; an output device; a processor; and a memory storing instructions executable by the processor. The instructions cause the processor to perform operations comprise: acquiring, by the input device, two-dimensional medical image data related to a prostate of a patient from a magnetic resonance imaging (MRI) device or a computed tomography (CT) device; generating, by the processor, three-dimensional voxel data of the prostate based on the two-dimensional medical image data; acquiring, by the input device, non-image clinical data including a serum prostate-specific antigen (PSA) level of the patient and a past diagnostic history of the patient; applying, by the processor, a feature extraction network to the three-dimensional voxel data to generate an imaging feature map, and generating a zone map identifying a peripheral zone (PZ) and a transition zone (TZ) of the prostate within the three-dimensional voxel data; determining, by the processor, a Biomarker-Adaptive Spatial Attention Strategy based on the non-image clinical data, wherein the strategy includes a zone sensitivity asymmetrically assigned to the peripheral zone (PZ) and the transition zone (TZ) according to the PSA level or the past diagnostic history, and a time-responsive weight according to a likelihood of lesion progression; operating, by the processor, a multimodal cross-attention module according to the determined strategy to dynamically modulate attention weights assigned to spatial locations within the imaging feature map; analyzing, by the processor, the modulated imaging feature map to detect an entire prostate region and one or more suspected cancer lesions, and calculating a total prostate volume and a lesion volume of the suspected cancer lesion; calculating, by the processor, a Tumor Burden Ratio, which is a ratio of the lesion volume to the total prostate volume, and a Corrected Prostate-Specific Antigen Density (Corrected PSAD) obtained by dividing the PSA level by a non-lesion prostate volume obtained by excluding the lesion volume from the total prostate volume; and outputting, by the output device, integrated diagnostic information including the Tumor Burden Ratio and the Corrected PSAD together with a determination result indicating whether the suspected cancer lesion corresponds to Clinically Significant Prostate Cancer (CSPC).

According to various embodiments of The present disclosure, there is provided a non-transitory computer-readable storage medium having stored thereon instructions that, when executed by at least one processor, cause the at least one processor to perform operations, wherein the operations comprise the steps of the method performed by the electronic device according to various embodiments of The present disclosure.

### BRIEF DESCRIPTION OF THE DRAWINGS

FIG. 1 illustrates an overview of an operation of an electronic device according to various embodiments of The present disclosure.

FIG. 2 illustrates a configuration of an electronic device according to various embodiments of The present disclosure.

FIG. 3 illustrates a method of operating an electronic device according to various embodiments of The present disclosure.

FIG. 3A illustrates a method of operating an electronic device according to various embodiments of The present disclosure.

FIG. 4 illustrates a structure of a multi-layer perceptron (MLP) for implementing a deep learning algorithm according to various embodiments of The present disclosure.

FIG. 5 illustrates an example of a preprocessing process of image data before inputting the image data to an artificial intelligence model according to various embodiments of The present disclosure.

FIG. 6 illustrates an example of an artificial intelligence model for detecting prostate cancer according to various embodiments of The present disclosure.

FIG. 7 illustrates an example of a 3D U-Net structure (MRI image input example) according to various embodiments of The present disclosure.

FIG. 8 illustrates an example of an output screen of an electronic device for providing determination information regarding clinically significant prostate cancer (CSPC) based on an artificial intelligence model according to various embodiments of The present disclosure.

FIG. 9 illustrates another example of an output screen of an electronic device for providing determination information regarding clinically significant prostate cancer (CSPC) based on an artificial intelligence model according to various embodiments of The present disclosure.

### DETAILED DESCRIPTION OF EXEMPLARY EMBODIMENTS

Hereinafter, embodiments of The present disclosure will be described in detail with reference to the accompanying drawings so that those skilled in the art may easily practice The present disclosure. It should be understood that The present disclosure may be embodied in different ways and is not limited to the following embodiments.

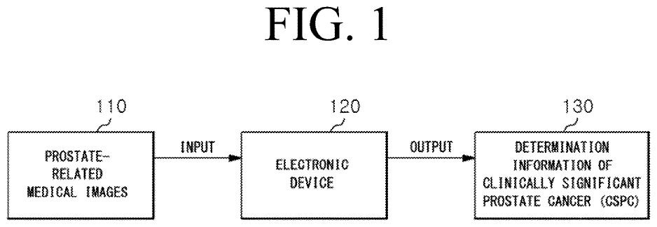

FIG. 1 illustrates an overview of an operation of an electronic device according to various embodiments of The present disclosure.

Referring to FIG. 1, an electronic device 120 according to various embodiments of The present disclosure operates in a structure in which prostate-related medical images 110 are received as input and determination information 130 indicating clinically significant prostate cancer (Clinically Significant Prostate Cancer, CSPC) is output.

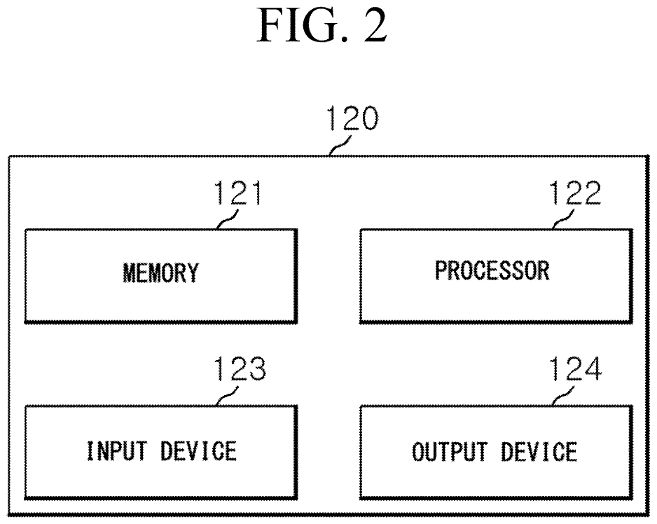

FIG. 2 illustrates a configuration of an electronic device according to various embodiments of The present disclosure.

Referring to FIG. 2, an electronic device 120 includes a memory 121, a processor 122, an input device 123, and an output device 124.

The memory 121 is connected to the processor 122, the input device 123, and the output device 124, and may store information input through the input device 123. The memory 121 may also be connected to the processor 122 and may store data such as a basic program for operation of the processor 122, application programs, configuration information, and information generated by operations of the processor 122. The memory 121 may include a volatile memory, a non-volatile memory, or a combination of a volatile memory and a non-volatile memory. The memory 121 may provide stored data in response to a request from the processor 122.

The processor 122 may be configured to implement procedures and/or methods proposed in The present disclosure. The processor 122 controls overall operations of the electronic device 120. For example, the processor 122 may write data to the memory 121 and read data from the memory 121. The processor 122 may also receive information through the input device 123 and output information through the output device 124. The processor 122 may include at least one processor.

The input device 123 may be connected to the processor 122 and may receive input information. The input device 123 may include a touch display, a keypad, a keyboard, or an information input module. According to an embodiment, the electronic device 120 may further include a transceiver, and the input device 123 may receive information from another device connected through a wired or wireless communication network via the transceiver.

The output device 124 may be connected to the processor 122 and may output information in the form of video, audio, or other forms. The output device 124 may include a display, a speaker, or an information output module. According to an embodiment, the electronic device 120 may further include a transceiver, and the output device 124 may transmit output information to another device connected through a wired or wireless communication network via the transceiver.

Although not shown in FIG. 2, according to an embodiment, the electronic device 120 may further include a transceiver. The transceiver may be connected to the processor 122 and may transmit and/or receive signals. All or part of the transceiver may be referred to as a transmitter, a receiver, or a transceiver. The transceiver may support at least one of various wireless communication standards including wired and wireless access systems such as an IEEE 802.xx system, an IEEE Wi-Fi system, a 3GPP system, a 3GPP LTE system, a 3GPP 5G NR system, a 3GPP 6G system, a 3GPP 2 system, and Bluetooth.

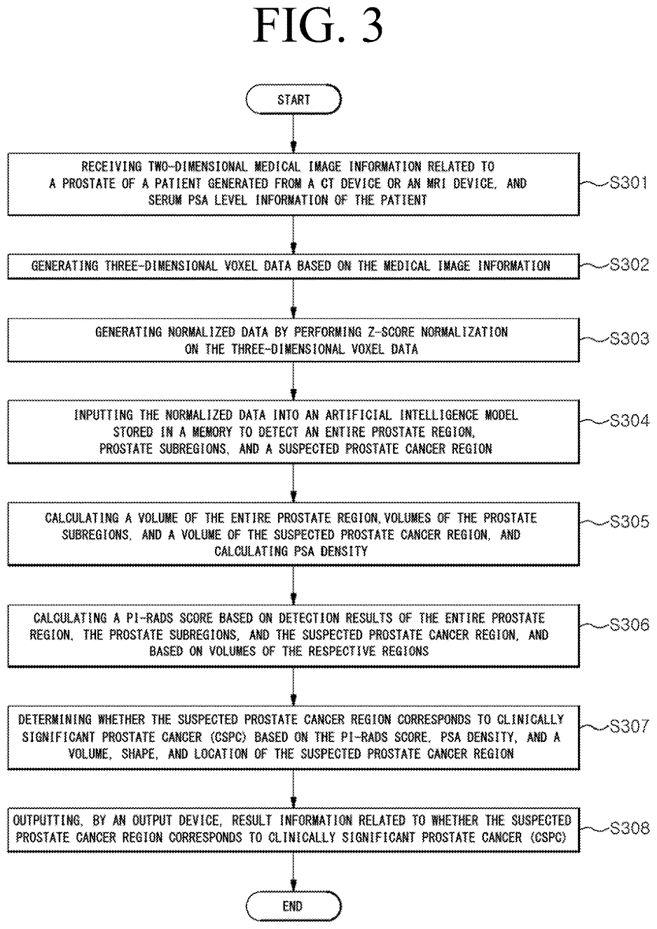

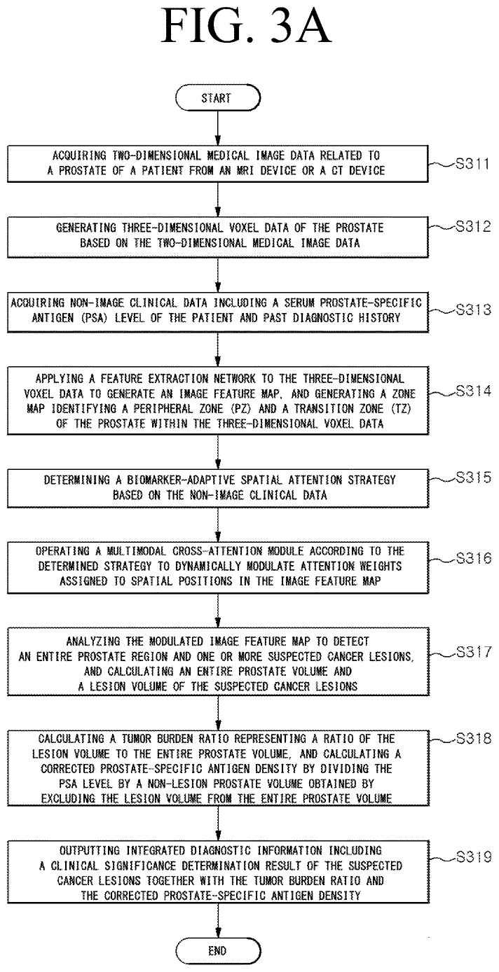

FIG. 3 illustrates a method of operating an electronic device according to various embodiments of The present disclosure.

Referring to FIG. 3, the embodiment of FIG. 3 illustrates an example of a method performed by an electronic device for providing determination information regarding clinically significant prostate cancer (Clinically Significant Prostate Cancer, CSPC) based on prostate images. The electronic device may include an input device, an output device, a processor, and a memory.

In step S301, the electronic device receives, through the input device, two-dimensional medical image information related to a prostate of a patient generated from a computed tomography (CT) device or a magnetic resonance imaging (MRI) device, and serum prostate-specific antigen (PSA) level information of the patient.

In step S302, the electronic device generates three-dimensional voxel data based on the medical image information using the processor.

In step S303, the electronic device performs Z-score normalization on the three-dimensional voxel data to generate normalized data using the processor.

In step S304, the electronic device inputs the normalized data into an artificial intelligence model stored in the memory and detects, using the processor, an entire prostate region, prostate subregions, and suspected prostate cancer regions.

In step S305, the electronic device calculates, using the processor, volumes of the entire prostate region, the prostate subregions, and the suspected prostate cancer regions, and prostate-specific antigen density (PSAD).

In step S306, the electronic device calculates, using the processor, a Prostate Imaging Reporting and Data System (PI-RADS) score based on detection results of the entire prostate region, the prostate subregions, and the suspected prostate cancer regions, and the calculated volumes of the respective regions.

In step S307, the electronic device determines, using the processor, whether the suspected prostate cancer region corresponds to clinically significant prostate cancer (CSPC) based on the PI-RADS score, the PSA density, and the volume, shape, and location of the suspected prostate cancer region.

The PI-RADS score, PSA density, and the volume, shape, and location of the suspected prostate cancer region are comprehensively evaluated for determining clinically significant prostate cancer (CSPC). The PI-RADS score is determined based on radiological analysis results derived from multiple sequences of prostate MRI, including T2-weighted imaging (T2WI), diffusion-weighted imaging (DWI), apparent diffusion coefficient (ADC), and dynamic contrast-enhanced MRI (DCE-MRI), and plays a key role in assessing the likelihood of malignancy of tumor lesions. A higher PI-RADS score indicates a higher likelihood of malignancy, thereby identifying prostate cancer suspected regions having a higher probability of CSPC. PSA density is calculated by dividing the serum PSA level by the total prostate volume and is used as an important auxiliary indicator for differentiating prostate cancer. In general, a PSA density of 0.15 ng/mL/cc or higher is considered indicative of a higher likelihood of prostate cancer and is evaluated together with the PI-RADS score. In addition, the volume, shape, and location of the suspected prostate cancer region also serve as important factors for determining CSPC. For example, when a lesion is large, exhibits an asymmetric shape, or lacks a clear boundary with surrounding tissues, the likelihood of malignancy is considered higher. By comprehensively analyzing these factors, it is finally determined whether the suspected prostate cancer region corresponds to CSPC.

In step S308, the electronic device outputs, through the output device, result information indicating whether the suspected prostate cancer region corresponds to CSPC.

According to various embodiments of The present disclosure, the three-dimensional voxel data may be generated by combining multiple two-dimensional image slices based on the medical image information.

According to various embodiments of The present disclosure, the suspected prostate cancer region may be detected by performing two-dimensional object detection on each of the multiple image slices based on the medical image information and integrating the detected two-dimensional suspected prostate cancer regions into three-dimensional masking data.

According to various embodiments of The present disclosure, the artificial intelligence model may be based on a convolutional neural network (CNN). The prostate subregions may include at least one of a transition zone (TZ), a peripheral zone (PZ), a central zone (CZ), and a seminal vesicle (SV). The artificial intelligence model may be trained to independently calculate volumes of the entire prostate region and one or more of the transition zone, peripheral zone, central zone, and seminal vesicle.

According to various embodiments of The present disclosure, the PSA density may be calculated based on the serum PSA level and the volume of the entire prostate region.

According to various embodiments of The present disclosure, the PI-RADS score may be calculated based on one or more of T2-weighted imaging (T2WI), diffusion-weighted imaging (DWI), apparent diffusion coefficient (ADC), and dynamic contrast-enhanced MRI (DCE-MRI).

According to various embodiments of The present disclosure, when the suspected prostate cancer region is located in the transition zone (TZ), the PI-RADS score may be calculated based on T2WI.

According to various embodiments of The present disclosure, when the suspected prostate cancer region is located in the peripheral zone (PZ), the PI-RADS score may be calculated based on DWI and ADC.

According to various embodiments of The present disclosure, when DWI or T2WI is unclear, the PI-RADS score may be additionally calculated based on DCE-MRI.

According to various embodiments of The present disclosure, step S302 of generating the three-dimensional voxel data based on the medical image information may include extracting resolution information of the multiple image slices and spacing information between the slices from metadata of the medical image information, determining a region of interest (ROI) based on a prostate-centered region in the multiple image slices, and generating the three-dimensional voxel data by correcting information within the ROI using a multi-level resampling algorithm.

According to various embodiments of The present disclosure, the Z-score normalization may remove outliers and noise from the three-dimensional voxel data by performing filtering that compares a global average and a local average for each pixel value of the three-dimensional voxel data. The normalized data may be divided into patches of the ROI by the processor. The divided patches of the normalized data may be assigned weighted priority values for training of the artificial intelligence model according to detection probabilities of the suspected prostate cancer region.

According to various embodiments of The present disclosure, the normalized data may be dynamically divided into patch units based on data size. To optimize the training speed of the artificial intelligence model, the normalized data may be divided into patch units and input into the artificial intelligence model using parallel processing. Among the divided patches, patches having a higher likelihood of including the suspected prostate cancer region may be preferentially input to the artificial intelligence model for training. Different priority weights may be assigned to respective patches to improve detection accuracy of the suspected prostate cancer region.

According to various embodiments of The present disclosure, patches having a higher likelihood of including the suspected prostate cancer region may be determined by scoring the inclusion likelihood of the suspected prostate cancer region in a specific region of interest (ROI) of the prostate based on texture heterogeneity and contrast.

According to various embodiments of The present disclosure, filtering may be configured to apply a combination of a Gaussian filter and an average filter to remove high-frequency noise, and the filtered normalized data may be input into the artificial intelligence model.

According to various embodiments of The present disclosure, the embodiment of FIG. 3 may further include receiving past information related to the prostate of the patient through the input device.

According to various embodiments of The present disclosure, the artificial intelligence model may be configured to simultaneously detect the suspected prostate cancer region and benign prostatic hyperplasia based on the normalized data.

According to various embodiments of The present disclosure, the result information may visually provide a change trend based on comparison with past information regarding changes in size and location of the suspected prostate cancer region.

According to various embodiments of The present disclosure, the result information may include volume information of the entire prostate region, the prostate subregions, and the suspected prostate cancer region, PSA density information, and two-dimensional or three-dimensional visualized graphical information corresponding to the respective regions.

According to various embodiments of The present disclosure, an electronic device for providing prostate substructure information may include an input device, an output device, a transceiver, a processor, and a memory, wherein the memory stores instructions for operations executed by the processor, and the operations may be configured to perform the method of operation of the electronic device according to the embodiment of FIG. 3.

According to various embodiments of The present disclosure, a non-transitory computer-readable storage media storing instructions that, when executed by at least one processor, cause the at least one processor to perform operations may be provided, wherein the operations include the method of operation of the electronic device according to the embodiment of FIG. 3.

FIG. 3A illustrates a method of operating an electronic device according to various embodiments of The present disclosure.

In the embodiment of FIG. 3A, the electronic device includes an input device, an output device, a processor, and a memory.

Referring to FIG. 3A, in step S311, the electronic device acquires, by the input device, two-dimensional medical image data related to a prostate of a patient from a magnetic resonance imaging (MRI) device or a computed tomography (CT) device.

In step S312, the electronic device generates, by the processor, three-dimensional voxel data of the prostate based on the two-dimensional medical image data.

In step S313, the electronic device acquires, by the input device, non-image clinical data including a serum prostate-specific antigen (PSA) level of the patient and a past diagnostic history of the patient.

In step S314, the electronic device applies, by the processor, a feature extraction network to the three-dimensional voxel data to generate an imaging feature map, and generates a zone map identifying a peripheral zone (PZ) and a transition zone (TZ) of the prostate within the three-dimensional voxel data.

In step S315, the electronic device determines, by the processor, a Biomarker-Adaptive Spatial Attention Strategy based on the non-image clinical data. The strategy includes a zone sensitivity asymmetrically assigned to the peripheral zone (PZ) and the transition zone (TZ) according to the PSA level or the past diagnostic history, and a time-responsive weight according to a likelihood of lesion progression.

In step S316, the electronic device operates, by the processor, a multimodal cross-attention module according to the determined strategy to dynamically modulate attention weights assigned to spatial locations within the imaging feature map.

In step S317, the electronic device analyzes, by the processor, the modulated imaging feature map to detect an entire prostate region and one or more suspected cancer lesions, and calculates a total prostate volume and a lesion volume of the suspected cancer lesion.

In step S318, the electronic device calculates, by the processor, a Tumor Burden Ratio, which is a ratio of the lesion volume to the total prostate volume, and a Corrected Prostate-Specific Antigen Density (Corrected PSAD) obtained by dividing the PSA level by a non-lesion prostate volume obtained by excluding the lesion volume from the total prostate volume.

In step S319, the electronic device outputs, by the output device, integrated diagnostic information including the Tumor Burden Ratio and the Corrected PSAD together with a determination result indicating whether the suspected cancer lesion corresponds to Clinically Significant Prostate Cancer (CSPC).

This aspect includes an overall pipeline for generating three-dimensional voxel data of a prostate from magnetic resonance imaging (MRI)-or computed tomography (CT)-based two-dimensional medical image data and generating an imaging feature map through a feature extraction network. This aspect combines a zone map identifying a peripheral zone (PZ) and a transition zone (TZ) of the prostate with a Biomarker-Adaptive Spatial Attention Strategy determined based on non-image clinical data.

This aspect has an effect of improving consistency of detection by emphasizing lesion candidates that are difficult to distinguish based only on image information according to a clinical context and zonal characteristics. This aspect has an effect of simultaneously providing composite indicators required for determining clinical significance, by calculating both the Tumor Burden Ratio and the Corrected PSAD.

According to various embodiments of The present disclosure, the non-image clinical data may further include, in addition to the serum prostate-specific antigen (PSA) level, structured fields corresponding to the past diagnostic history. The structured fields may include at least one of information on past examination time points, summary information of past interpretation results or pathological results, information on a location of a past lesion or a zone assignment of the past lesion, and information indicating whether treatment corresponding to a prostate cancer treatment history has been performed or a type of the treatment. The feature extraction network may include a neural network configured to generate the imaging feature map by extracting voxel-level spatial features from the three-dimensional voxel data. The zone map may include, for each voxel of the three-dimensional voxel data, a zone label or a zone membership value indicating whether the voxel belongs to the peripheral zone (PZ) or the transition zone (TZ) of the prostate, and the zone membership value may include probability information or membership information represented as a continuous value. The Biomarker-Adaptive Spatial Attention Strategy may include elements configured to (i) set a first zone sensitivity applied to spatial locations corresponding to the peripheral zone (PZ) and a second zone sensitivity applied to spatial locations corresponding to the transition zone (TZ) differently from each other, and (ii) set a time-responsive weight such that spatial locations having a higher likelihood of lesion occurrence, expansion, or progression are relatively emphasized according to a temporal order of the past diagnostic history or a prostate cancer treatment history included in the non-image clinical data. The multimodal cross-attention module may include an attention operation configured to compute the attention weights using a clinical query vector generated by encoding the non-image clinical data as a query and the imaging feature map as keys and values. Dynamically modulating the attention weights may include adjusting a distribution of the attention weights assigned to spatial locations within the imaging feature map so that the distribution is changed according to zones or according to the time-responsive weight based on a value of the clinical query vector or the zone map. The suspected cancer lesion may be represented as a lesion candidate region identified based on the modulated imaging feature map and may be expressed as at least one of a lesion-instance mask or a lesion candidate probability map. The Tumor Burden Ratio may include a ratio of the lesion volume of the suspected cancer lesion to the total prostate volume or a ratio of a sum of lesion volumes of suspected cancer lesions to the total prostate volume. The non-lesion prostate volume may include a volume of remaining voxels obtained by excluding voxels included in the suspected cancer lesion from voxels constituting the entire prostate region. The Corrected Prostate-Specific Antigen Density (Corrected PSAD) may include a value obtained by dividing the PSA level by the non-lesion prostate volume.

This aspect is directed to a form in which the non-image clinical data include, in addition to the PSA level, structured fields corresponding to the past diagnostic history, including past examination time points, summary information of interpretation results or pathological results, information on a location of a past lesion or a zone assignment of the past lesion, and information indicating whether treatment corresponding to a prostate cancer treatment history has been performed or a type of the treatment. This aspect is directed to a form including an attention operation structure in which the clinical query vector is a vector generated by encoding the non-image clinical data and is used as a query in the multimodal cross-attention module, and the imaging feature map is used as keys and values.

This aspect has an effect in that modulation of the attention weights is directly linked to clinical evidence because items, time points, and summary values of clinical information are reflected in a structured manner. This aspect has an effect of increasing comparability among patients because the Tumor Burden Ratio encompasses either a single lesion volume or a sum of volumes of multiple lesions, and the Corrected Prostate-Specific Antigen Density is configured to be corrected using the non-lesion prostate volume.

According to various embodiments of The present disclosure, the non-image clinical data may include prostate-specific antigen (PSA) trend information at multiple time points. The PSA trend information may include a time-series array of PSA values corresponding to respective time points, examination time-point metadata corresponding to the time-series array, and at least one of a change-direction feature, a change-rate feature, a change-acceleration feature, and a sudden-change event flag generated from the time-series array. The time-responsive weight may be configured as a spatially varying time-weight map in which different temporal weights are assigned to spatial locations according to at least one of the change-direction feature, the change-rate feature, the change-acceleration feature, and the sudden-change event flag.

This aspect is directed to a form in which PSA trend information at multiple time points includes a time-series array, examination time-point metadata, a change-direction feature, a change-rate feature, a change-acceleration feature, and a sudden-change event flag. This aspect is directed to a form in which the time-responsive weight is configured as a spatially varying time-weight map, so that temporal change characteristics are combined with spatial emphasis.

This aspect has an effect in that sensitivity of lesion exploration is adjusted in patients having temporally meaningful changes by reflecting even the rate and acceleration of PSA change patterns. This aspect has an effect in that, even for the same image, an emphasis distribution changes according to a clinical context at each time point, thereby improving consistency of change detection in follow-up observation.

According to various embodiments of The present disclosure, the Biomarker-Adaptive Spatial Attention Strategy may be configured as a strategy parameter packet including (i) a zone weight parameter set representing a relative relationship between a first zone weight corresponding to the peripheral zone (PZ) and a second zone weight corresponding to the transition zone (TZ), and (ii) a spatial distribution parameter set representing a distribution of spatial locations to which the time-responsive weight is applied, the strategy parameter packet being generated based on a combination function using, as inputs, a clinical risk context vector generated from the non-image clinical data and a zone context vector generated from the zone map.

This aspect is directed to a form in which a strategy parameter packet is generated based on a combination function using, as inputs, a clinical risk context vector and a zone context vector. This aspect is directed to a form in which the strategy parameter packet includes a zone weight parameter set and a spatial distribution parameter set, thereby together including zone asymmetry and a spatial application range of the time-responsive weight.

This aspect has an effect in that reproducibility of the strategy is improved in repeated execution or model update processes because the strategy is managed as a parameter bundle rather than a textual instruction. This aspect has an effect in that zone-specific sensitivity and spatial distribution are controlled at the same time, thereby suppressing false positives biased toward a zone boundary or a particular zone while stably emphasizing clinically important regions.

According to various embodiments of The present disclosure, the past diagnostic history may include at least one of a center coordinate of a lesion candidate, zone assignment information of the lesion candidate, and size information or size change information of the lesion candidate obtained from a past imaging examination or a past interpretation result. The time-responsive weight may be configured as a location-specific time-weight map reflecting a likelihood of lesion occurrence, expansion, or progression by being combined with a spatial prior map generated based on at least one of the center coordinate of the lesion candidate and the zone assignment information.

This aspect is directed to a form in which the past diagnostic history includes a center coordinate of a lesion candidate, zone assignment information of the lesion candidate, and size information or size change information of the lesion candidate. This aspect is directed to a form in which the time-responsive weight is combined with a spatial prior map to form a location-specific time-weight map, so that a past lesion hint is reflected in current image emphasis.

This aspect has an effect of increasing stability of exploration at positions having a higher likelihood of recurrence or progression in follow-up observation by using a location and zone assignment of a past lesion. This aspect has an effect of generating an emphasis distribution reflecting not merely a remembered position but also a change tendency because past lesion size change information is combined therewith.

According to various embodiments of The present disclosure, the multimodal cross-attention module may generate a Key component and a Value component from the imaging feature map and may generate a Query component from the clinical query vector. The zone sensitivity may be configured as a zone-specific scaling tensor applied along a channel axis of the Key component or the Value component. The zone-specific scaling tensor may be masked by a zone membership channel of the zone map so that different scaling factors are applied to the peripheral zone (PZ) and the transition zone (TZ).

This aspect is directed to a form in which the multimodal cross-attention module generates a Key component, a Value component, and a Query component, respectively. This aspect is directed to a form in which the zone sensitivity is configured as a zone-specific scaling tensor and is masked by a zone membership channel so that different scaling factors are applied to the peripheral zone (PZ) and the transition zone (TZ).

This aspect has an effect in that, even for the same imaging feature, a different amplification or attenuation is applied for each zone, so that a sensitivity difference based on zonal characteristics is directly reflected in an attention operation. This aspect has an effect in that, because a masking structure is included, gradual scaling change is possible according to continuous membership values even at a zone boundary or a mixed region, thereby reducing unstable determinations in the vicinity of the boundary.

According to various embodiments of The present disclosure, an evidence map may include a voxel-level contribution map generated based on a spatial distribution of the attention weights. The evidence map may include a peripheral-zone evidence map and a transition-zone evidence map based on the zone map. The integrated diagnostic information may further include display information in which the peripheral-zone evidence map and the transition-zone evidence map are overlaid on the medical image data as distinguishable layers.

This aspect is directed to a form in which the evidence map includes a voxel-level contribution map generated based on a spatial distribution of the attention weights. This aspect is directed to a form in which the evidence map includes a peripheral-zone evidence map and a transition-zone evidence map, and the integrated diagnostic information includes layer overlay display information.

This aspect has an effect in that a diagnostic result is presented not only as a single numerical value but also together with contribution-based visual evidence, thereby increasing clinical interpretability. This aspect has an effect in that, because zone-specific evidence maps are separately provided, it is possible to identify by which zonal characteristic the same lesion has been emphasized, thereby increasing explanatory power and verifiability.

According to various embodiments of The present disclosure, the attention weights may include zone-wise normalized attention distributions obtained by applying independent normalization operations to a peripheral-zone (PZ) domain and a transition-zone (TZ) domain separated by the zone map. The zone-wise normalized attention distributions may be configured such that normalization contributions of boundary-adjacent voxels are adjusted based on continuous zone membership values and boundary uncertainty information included in the zone map.

This aspect is directed to a form in which the attention weights are configured as zone-wise normalized attention distributions obtained by applying independent normalization operations in a peripheral-zone (PZ) domain and a transition-zone (TZ) domain, respectively. This aspect is directed to a form in which normalization contributions of boundary-adjacent voxels are adjusted using continuous zone membership values and boundary uncertainty information.

This aspect has an effect in that, even when signal distributions differ among zones, a phenomenon in which one zone excessively dominates is reduced by the same normalization criterion. This aspect has an effect in that contributions of voxels having high boundary uncertainty are adjusted, thereby reducing unstable emphasis or unnecessary false positives occurring at a zone boundary. The zone-wise normalization operation modifies a normalization domain of an attention distribution so that independent attention distributions are generated for the peripheral zone (PZ) and the transition zone (TZ).

According to various embodiments of The present disclosure, the non-lesion prostate volume may be calculated using a non-lesion weight map generated based on voxel-wise lesion probability values of a probability map for the suspected cancer lesion. The non-lesion weight map may be configured as a zone-adaptive non-lesion weight map in which different weighting functions are applied for respective zones according to a zone membership channel of the zone map. An expected non-lesion volume may be calculated by a weighted integration of a product of the zone-adaptive non-lesion weight map and voxel-wise volumes of the entire prostate region.

This aspect is directed to a form in which the non-lesion prostate volume is calculated using a non-lesion weight map. This aspect is directed to a form in which the non-lesion weight map is configured as a zone-adaptive non-lesion weight map in which different weighting functions are applied for respective zones according to a zone membership channel, and the expected non-lesion volume is calculated by weighted integration.

This aspect has an effect in that a non-lesion contribution of voxels having a high lesion probability is naturally lowered, so that a denominator of the Corrected Prostate-Specific Antigen Density is calculated more stably. This aspect has an effect in that sensitivity of non-lesion estimation can be differently set for each zone, thereby reducing distortion of an estimated normal volume according to zonal characteristics.

According to various embodiments of The present disclosure, the non-lesion prostate volume may be calculated as a sum of voxel-wise volumes of remaining voxels obtained by excluding voxels corresponding to the suspected cancer lesion from voxels constituting the entire prostate region. The voxels corresponding to the suspected cancer lesion may be identified by at least one of a lesion-instance mask and a lesion probability map of the suspected cancer lesion.

This aspect is directed to a form in which the non-lesion prostate volume is calculated as a sum of voxel-wise volumes of remaining voxels obtained by excluding voxels corresponding to the suspected cancer lesion from voxels of the entire prostate region. This aspect includes a form in which the voxels corresponding to the suspected cancer lesion are identified by a lesion-instance mask or a lesion probability map.

This aspect has an effect in that a basis for calculating the non-lesion prostate volume becomes clear as a voxel set and an identification scheme, thereby improving consistency of calculation of the Corrected Prostate-Specific Antigen Density. This aspect has an effect of maintaining logic for calculating an estimated normal volume even when an output format of a lesion changes, because both a mask-based form and a probability map-based form are encompassed.

According to various embodiments of The present disclosure, the non-image clinical data may include, in addition to the serum prostate-specific antigen (PSA) level and the past diagnostic history, structured fields corresponding to at least one of an age of the patient, a prostate volume measurement history, a clinical history related to prostatitis, a clinical history related to benign prostatic hyperplasia, family history information, and medication administration history. The structured fields may include an item identifier and item-specific measurement time-point metadata.

This aspect is directed to a form in which the non-image clinical data include structured fields corresponding to age, prostate volume measurement history, clinical history related to prostatitis, clinical history related to benign prostatic hyperplasia, family history, and medication administration history. This aspect is directed to a form in which the structured fields include an item identifier and item-specific measurement time-point metadata.

This aspect has an effect in that meaning and timing of clinical items are explicitly combined, so that temporal-axis information of the same patient is stably reflected in a clinical query vector encoding process. This aspect has an effect in that confounding factors such as inflammation, hyperplasia, and medication are expressed together, so that image emphasis is adjusted according to a patient context.

According to various embodiments of The present disclosure, the medical image data may be multi-parametric MRI (mpMRI) data including T2-weighted imaging (T2WI), diffusion-weighted imaging (DWI), and an apparent diffusion coefficient (ADC) map. The imaging feature map may include feature channels corresponding to respective parameters of the multi-parametric MRI data and a fused feature channel including a complementary feature combination component among the feature channels corresponding to the respective parameters.

This aspect is directed to a form in which the medical image data are multi-parametric MRI (mpMRI) data including T2-weighted imaging (T2WI), diffusion-weighted imaging (DWI), and an apparent diffusion coefficient (ADC) map. This aspect is directed to a form in which the imaging feature map includes feature channels corresponding to respective parameters and a fused feature channel including a complementary feature combination component among parameters.

This aspect has an effect in that both structural cues and diffusion cues of a lesion are reflected together as compared with a single-sequence-based model, thereby increasing expressiveness of a lesion candidate. This aspect has an effect in that, because an inter-parameter combination component is included, pieces of information having different image contrasts operate complementarily, thereby improving discriminability of an ambiguous lesion.

According to various embodiments of The present disclosure, the multimodal cross-attention module may include a plurality of attention weight distributions calculated by a plurality of attention heads. The plurality of attention weight distributions may include head group identification information corresponding to a peripheral-zone (PZ) domain and a transition-zone (TZ) domain separated by the zone map, respectively. The head group identification information may be associated with the zone-specific scaling tensor or the zone-wise independent normalization operation.

This aspect is directed to a form in which the multimodal cross-attention module includes a plurality of attention weight distributions, and each distribution is associated with head group identification information. This aspect includes a form in which the head group identification information is associated with the zone-specific scaling tensor or the zone-wise independent normalization operation so that different attention components are managed for respective zonal domains.

This aspect has an effect in that attention distributions become interpretable from a viewpoint separated for respective zones, thereby facilitating cause analysis of zonal bias and model tuning. This aspect has an effect in that zone-wise operations and head groups are combined, so that an attention representation suitable for zonal characteristics is stably maintained.

According to various embodiments of The present disclosure, the suspected cancer lesion may include, for each lesion instance, a center location coordinate, an uncertainty score corresponding to a lesion boundary region, and lesion-instance-specific zone assignment information calculated based on the zone map. The lesion-instance-specific zone assignment information may include zone-wise aggregated statistics based on zone membership values of voxels within a lesion-instance mask.

This aspect is directed to a form in which the suspected cancer lesion includes, for each lesion instance, a center location coordinate, an uncertainty score corresponding to a lesion boundary region, and lesion-instance-specific zone assignment information. This aspect is directed to a form in which the lesion-instance-specific zone assignment information includes zone-wise aggregated statistics based on zone membership values of voxels within a mask.

This aspect has an effect in that lesion location and zone assignment are provided in quantified form, thereby enabling a clinician to quickly determine by which zonal characteristic the lesion has been presented. This aspect has an effect in that, because boundary uncertainty is also provided, reliability of lesion volume or location determination is taken into consideration together.

According to various embodiments of The present disclosure, the determination result may include a set of lesion-instance-specific malignancy probabilities and a patient-level composite risk value generated based on at least one of the set of lesion-instance-specific malignancy probabilities and the Tumor Burden Ratio. The patient-level composite risk value may include a result of a combination operation of at least one of a maximum value, an average value, and a weighted sum of the set of lesion-instance-specific malignancy probabilities, and the Tumor Burden Ratio.

This aspect is directed to a form in which the determination result includes a set of lesion-instance-specific malignancy probabilities and a patient-level composite risk value. This aspect is directed to a form in which the patient-level composite risk value includes a result of a combination operation of at least one of a maximum value, an average value, and a weighted sum, and the Tumor Burden Ratio.

This aspect has an effect in that, even when multiple lesions are present, lesion-level information and a patient-level conclusion are provided together, thereby simplifying a clinical decision-making flow. This aspect has an effect in that, because the Tumor Burden Ratio is combined, risk stratification reflecting lesion burden is possible even when the same malignancy probability is present.

According to various embodiments of The present disclosure, the clinical query vector may include a set of item-specific embedding vectors of the non-image clinical data and a temporal order encoding indicating an order of time points of the past diagnostic history. The set of item-specific embedding vectors may include mask information indicating presence or absence of respective items and an embedding component of a missing-indication token indicating missingness of each item.

This aspect is directed to a form in which the clinical query vector includes a set of item-specific embedding vectors and a temporal order encoding. This aspect is directed to a form in which the mask information indicating presence or absence of respective items and the embedding component of the missing-indication token are included, so that omission of a clinical item is directly reflected in a vector representation.

This aspect has an effect in that missingness is represented not as a simple blank but as an explicit token, so that an influence of missingness is maintained in a controllable form in an attention operation. This aspect has an effect in that, because an item presence mask is included, it is easy to extend an item-specific reliability or weighting policy.

According to various embodiments of The present disclosure, the integrated diagnostic information may be configured in a form of a structured report. The structured report may include at least one of a lesion-instance-specific summary field, a zone-specific summary field, and a time-series summary field. The lesion-instance-specific summary field may include lesion-instance-specific location information, lesion-instance-specific zone assignment information, and lesion-instance-specific malignancy probability. The zone-specific summary field may include a zone-specific Tumor Burden Ratio component for each of the peripheral zone (PZ) and the transition zone (TZ).

This aspect is directed to a form in which the integrated diagnostic information is configured in a form of a structured report and includes at least one of a lesion-instance-specific summary field, a zone-specific summary field, and a time-series summary field. This aspect is directed to a form in which the lesion-instance-specific summary field includes location, zone assignment, and malignancy probability, and the zone-specific summary field includes a Tumor Burden Ratio component for each of the peripheral zone and the transition zone.

This aspect has an effect in that, because output is provided on a field basis rather than as free text, linkage with a hospital system and comparison in follow-up observation are facilitated. This aspect has an effect in that, because a zone-specific summary is included, risk signals according to zonal characteristics are interpreted separately even for the same patient.

According to various embodiments of The present disclosure, when some items of the non-image clinical data are missing, the clinical query vector may include an embedding component of a missing-indication token corresponding to the missing items. The attention weights calculated by the multimodal cross-attention module may be configured such that a contribution of an attention component corresponding to item missingness is adjusted based on the embedding component of the missing-indication token.

This aspect is directed to a form in which, when some items of the non-image clinical data are missing, the clinical query vector includes the embedding component of the missing-indication token. This aspect is directed to a form in which the attention weights are configured such that a contribution of an attention component corresponding to missingness is adjusted based on the embedding component of the missing-indication token.

This aspect has an effect in that, even in patient data having missing values, a phenomenon in which attention excessively depends on a specific item is reduced. This aspect has an effect in that, because an operation in a missing-data situation is explicitly defined, variability of model output is reduced and prediction stability in clinical application is improved.

According to various embodiments of The present disclosure, the Tumor Burden Ratio may include an overall Tumor Burden Ratio component that is a ratio relative to the total prostate volume. The Tumor Burden Ratio may further include a zone-specific Tumor Burden Ratio component calculated, for each of the peripheral zone (PZ) and the transition zone (TZ) separated by the zone map, as a ratio of a zone-specific lesion volume to a zone-specific volume. The zone-specific lesion volume may be calculated based on a combination of a lesion-instance mask of the suspected cancer lesion and the zone membership channel.

This aspect is directed to a form in which the Tumor Burden Ratio includes an overall Tumor Burden Ratio component and further includes a zone-specific Tumor Burden Ratio component. This aspect is directed to a form in which the zone-specific lesion volume is calculated based on a combination of the lesion-instance mask and the zone membership channel.

This aspect has an effect in that, because not only an overall burden but also zone-specific burden is calculated together, risk assessment reflecting clinical differences between the peripheral zone and the transition zone becomes possible. This aspect has an effect in that, because continuous membership is combined, zone-specific volume calculation is stably performed even for a lesion crossing a zone boundary.

According to various embodiments of the present disclosure, the multimodal cross-attention module may be further configured to generate a voxel-level contribution tensor by back-projecting the attention weights from spatial positions of the imaging feature map to voxel coordinates of the three-dimensional voxel data. The voxel-level contribution tensor may be partitioned, based on zone membership information included in the zone map, into a peripheral-zone contribution tensor corresponding to the peripheral zone (PZ) and a transition-zone contribution tensor corresponding to the transition zone (TZ). The integrated diagnostic information may further include lesion-instance-specific zonal evidence scores generated by accumulating voxel-level contributions of the voxel-level contribution tensor within a lesion-instance mask corresponding to the suspected cancer lesion.

This aspect is directed to a configuration in which attention weights calculated in the multimodal cross-attention module are not only used for lesion detection but are also transformed into voxel-level contribution information through a back-projection operation from feature-space positions to voxel coordinates of the three-dimensional voxel data. In this configuration, the voxel-level contribution tensor represents a spatial contribution distribution indicating how strongly each voxel contributes to the attention-weighted analysis result. The voxel-level contribution tensor may be decomposed according to zone membership information of the zone map into a peripheral-zone contribution tensor and a transition-zone contribution tensor. In addition, contributions included within a lesion-instance mask corresponding to the suspected cancer lesion may be accumulated to generate lesion-instance-specific zonal evidence scores.

This aspect has an effect in that attention information generated in a feature-space domain is explicitly mapped back to voxel coordinates of the three-dimensional voxel data, thereby improving spatial interpretability of attention operations. This aspect has an effect in that contributions originating from the peripheral zone (PZ) and the transition zone (TZ) are separately quantified, thereby enabling a user to identify which zonal characteristic predominantly supports the lesion detection result. This aspect has an effect in that lesion-instance-specific zonal evidence scores are generated, thereby providing quantitative explanatory information supporting the integrated diagnostic information.

According to various embodiments of the present disclosure, the attention weights may be normalized using a dual-domain normalization operation in which a first normalization is performed over spatial locations belonging to a peripheral-zone domain and a second normalization is performed over spatial locations belonging to a transition-zone domain, the peripheral-zone domain and the transition-zone domain being defined by the zone map. Boundary-adjacent voxels located near a boundary between the peripheral-zone domain and the transition-zone domain may be assigned fractional domain participation coefficients based on continuous zone membership values included in the zone map. The dual-domain normalization operation may further apply a boundary suppression coefficient to a boundary-adjacent voxel having boundary uncertainty information exceeding a predetermined threshold so as to reduce dominance of the boundary-adjacent voxel in both the first normalization and the second normalization.

This aspect is directed to a configuration in which the attention weights are normalized separately for different zonal domains defined by the zone map. In this configuration, the peripheral-zone domain includes spatial locations corresponding to the peripheral zone (PZ), and the transition-zone domain includes spatial locations corresponding to the transition zone (TZ). The dual-domain normalization operation may generate independent attention distributions for the respective domains. Voxels located near a boundary between the zones may have fractional domain participation coefficients determined based on continuous zone membership values. In addition, a boundary suppression coefficient may be applied when boundary uncertainty information exceeds a predetermined threshold so that boundary-adjacent voxels do not excessively dominate attention distributions in either domain.

This aspect has an effect in that attention normalization is performed independently within zonal domains, thereby preventing an imbalance in which a dominant zone disproportionately influences the overall attention distribution. This aspect has an effect in that boundary-adjacent voxels having high uncertainty are prevented from simultaneously dominating attention distributions of multiple zones. This aspect has an effect in that zonal characteristics of prostate anatomy are explicitly incorporated into the attention normalization process, thereby improving stability of lesion detection near zone boundaries.

According to various embodiments of the present disclosure, the non-lesion prostate volume may be calculated as an expected non-lesion volume obtained by integrating voxel-wise non-lesion weights over voxels included in the entire prostate region. Each of the voxel-wise non-lesion weights may be computed as a complementary probability value with respect to a lesion probability assigned to a corresponding voxel of a lesion probability map representing the suspected cancer lesion and may be further modulated by a zone-adaptive correction coefficient determined from the zone map. The zone-adaptive correction coefficient may be differently defined for voxels belonging to the peripheral zone (PZ) and voxels belonging to the transition zone (TZ). The Corrected Prostate-Specific Antigen Density (Corrected PSAD) may be calculated using the expected non-lesion volume.

This aspect is directed to a configuration in which the non-lesion prostate volume is not calculated through a binary exclusion of lesion voxels but is estimated as an expected non-lesion volume based on probabilistic lesion information. In this configuration, a lesion probability map representing the suspected cancer lesion provides voxel-wise lesion probabilities, and voxel-wise non-lesion weights are calculated as complementary probabilities corresponding to the lesion probabilities. The voxel-wise non-lesion weights may be further adjusted using zone-adaptive correction coefficients derived from the zone map so that different weighting behaviors are applied to voxels belonging to the peripheral zone (PZ) and the transition zone (TZ). The expected non-lesion volume may then be obtained through weighted integration of the voxel-wise non-lesion weights across the entire prostate region.

This aspect has an effect in that uncertainty of lesion probability values is reflected in the estimation of the non-lesion prostate volume, thereby improving robustness of the denominator used for calculating the Corrected Prostate-Specific Antigen Density (Corrected PSAD). This aspect has an effect in that different correction behaviors may be applied to voxels belonging to the peripheral zone (PZ) and the transition zone (TZ), thereby reducing distortion caused by zone-specific false-positive tendencies. This aspect has an effect in that the Corrected PSAD reflects both probabilistic lesion information and zonal characteristics, thereby improving clinical interpretability of the integrated diagnostic information.

According to various embodiments of The present disclosure, an electronic device for providing prostate substructure information may include an input device, an output device, a transceiver, a processor, and a memory, wherein the memory stores instructions for operations executed by the processor, and the operations may be configured to perform the method of operation of the electronic device according to the embodiment of FIG. 3A.

According to various embodiments of The present disclosure, a non-transitory computer-readable storage media storing instructions that, when executed by at least one processor, cause the at least one processor to perform operations may be provided, wherein the operations include the method of operation of the electronic device according to the embodiment of FIG. 3A.

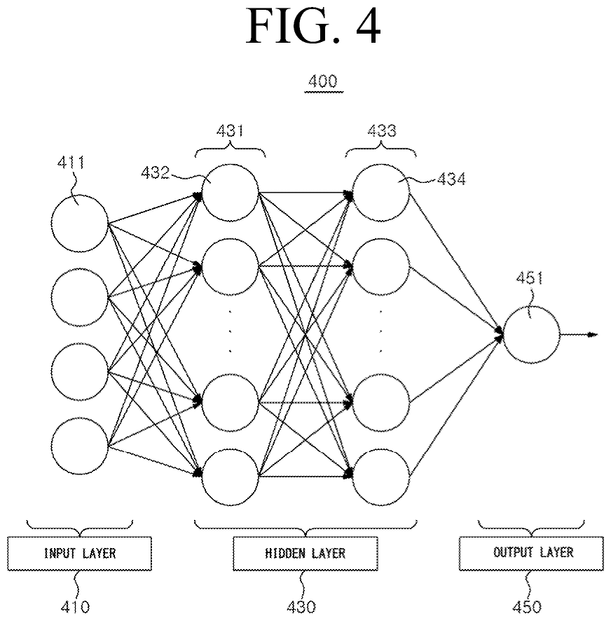

FIG. 4 illustrates a structure of a multi-layer perceptron (MLP) for implementing a deep learning algorithm according to various embodiments of The present disclosure.

According to various embodiments of The present disclosure, a prostate cancer suspected region analysis model may be configured in an MLP structure according to the embodiment of FIG. 4. The prostate cancer suspected region analysis model may be implemented using various architectures such as a convolutional neural network (CNN), DenseNet, U-Net, GoogLeNet, and a generative adversarial network. For example, when the prostate cancer suspected region analysis model is implemented as a CNN, the prostate cancer suspected region analysis model performs a training process for adjusting connection weights of an artificial neural network using training data.

Deep learning is one of the technologies that has recently emerged in the field of machine learning, and is a neural network composed of a plurality of hidden layers and a plurality of hidden units included therein. When low-level features are input to a deep learning model, the low-level features are transformed into high-level features that can better describe a problem to be predicted while passing through the plurality of hidden layers. Since expert prior knowledge or intuition is not required in this process, subjective factors in feature extraction can be eliminated, and a model having higher generalization capability can be developed. Furthermore, in the case of deep learning, since feature extraction and model construction are configured as one set, there is an advantage in that a final model can be formed through a simpler process as compared with conventional machine learning techniques.

A multi-layer perceptron (MLP) is a type of artificial neural network (ANN) having multiple nodes based on deep learning. Each node is a neuron similar to a connection pattern of an animal and uses a nonlinear activation function. This nonlinear property enables linearly separable classification of data that cannot otherwise be separated linearly.

Referring to FIG. 4, an artificial neural network 400 of an MLP model according to various embodiments of The present disclosure includes an input layer 410, a plurality of hidden layers 430, and an output layer 450.

Input data, such as a plurality of factors related to medical images, are input to nodes of the input layer 410. Here, the plurality of factors 411 related to medical images correspond to low-level features of the deep learning model.

At nodes of the hidden layers 430, calculations based on the input factors are performed. The hidden layers 430 are layers in which units defined as a plurality of nodes formed by combining the plurality of factors 411 related to medical images are stored. As illustrated in FIG. 4, the hidden layers 430 may include a plurality of hidden layers.

For example, when the hidden layers 430 include a first hidden layer 431 and a second hidden layer 433, the first hidden layer 431 is a layer in which first units 432 defined as a plurality of nodes formed by combining the plurality of factors 411 related to medical images, which are the lowest-level features, are stored, and each first unit 432 corresponds to a higher-level feature of the plurality of factors related to medical images. The second hidden layer 433 is a layer in which second units 434 defined as a plurality of nodes formed by combining the first units of the first hidden layer 431 are stored, and each second unit 434 corresponds to a higher-level feature of the first units 432.

Nodes of the output layer 450 represent calculated prediction results. The output layer 450 may include a plurality of prediction result units 451. More specifically, the plurality of prediction result units 451 may include two units, that is, a True unit and a False unit. More specifically, the True unit is a prediction result unit indicating that a medical image is related to a particular feature, and the False unit is a prediction result unit indicating that the medical image is not related to the particular feature.

Respective weights are assigned to connections between the second units 434 included in the second hidden layer 433, which is the last layer among the hidden layers 430, and the prediction result units 451. Based on these weights, whether the medical image is related to a particular feature is predicted.

For example, when one of the second units 434 predicts that a medical image is related to a particular feature, the unit is connected to the True unit and the False unit, respectively, and a positive weight is assigned to the connection to the True unit, while a negative weight is assigned to the connection to the False unit. Conversely, when one of the second units 434 predicts that the medical image is not related to the particular feature, the unit is connected to the True unit and the False unit, respectively, and a negative weight is assigned to the connection to the True unit, while a positive weight is assigned to the connection to the False unit.

A plurality of connection lines may be formed between the plurality of second units 434 and the True unit. When a total sum of the plurality of connection lines has a positive value, the plurality of factors 411 related to the medical image in the input layer 410 are predicted as factors indicating that the medical image is related to a particular feature. According to an embodiment, whether such a medical image is likely to be related to the particular feature may be predicted by comparing the total sum of the plurality of connection lines with a preset value.

The artificial neural network 400 of the MLP model is trained by adjusting training parameters. According to an embodiment, the training parameters include at least one of weights and biases. The training parameters are repeatedly adjusted through an optimization algorithm called gradient descent. Whenever a prediction result is calculated from a given data sample, that is, during forward propagation, performance of the network is evaluated through a loss function that measures prediction error. Each training parameter of the artificial neural network 400 is adjusted by being incrementally changed in a direction that minimizes a value of the loss function, and this process is referred to as back-propagation.

The present disclosure is directed to a software program for analyzing CT and MRI images, measuring a prostate volume, detecting a suspected prostate cancer region, calculating a PI-RADS score, and determining whether clinically significant prostate cancer (CSPC) is present, with the purpose of assisting a user in performing a CSPC diagnosis more efficiently.

The present disclosure supports DICOM and NIFTI standards, which are mainly used in medical devices and PACS (Picture Archiving and Communication System) software, and intuitively provides a user with a prostate volume, a suspected cancer region, and a CSPC determination result by visualizing multi-slice medical images in two-dimensional and three-dimensional forms.

The present disclosure also calculates prostate-specific antigen density (PSAD) based on a prostate volume in cubic centimeters and a serum PSA level in ng/mL, and the PSAD is used as an auxiliary indicator for determining whether CSPC is present based on information such as a PI-RADS score and a volume, shape, and location of a suspected prostate cancer region.

The present disclosure supports a user in diagnosing clinically significant prostate cancer more accurately and rapidly by automating a process of measuring a prostate volume, detecting a suspected cancer region, calculating a PI-RADS score, and determining whether CSPC is present.

The software is composed of four artificial intelligence models, each of which is responsible for measuring an entire prostate volume, measuring volumes of prostate subregions including a transition zone, a peripheral zone, and a central zone, detecting a suspected prostate cancer region, calculating a PI-RADS score, and determining CSPC. The entire prostate volume is used for calculating PSA density, and the measurement of the subregions is used to evaluate whether benign prostatic hyperplasia is present. Detection of the suspected prostate cancer region visually indicates a cancer-suspected site in a plurality of CT and MRI images, and finally a PI-RADS score calculation and CSPC determination model evaluates whether the corresponding region corresponds to clinically significant prostate cancer and provides the evaluation to the user.

Prostate cancer is a disease in which the number of patients has been steadily increasing due to aging and the spread of Western-style dietary habits. According to the Health Insurance Review and Assessment Service of Korea, the number of prostate cancer patients increased by about 41.56%, from 85,943 in 2018 to 121,665 in 2022, and medical expenses increased by 52.8% during the same period. This further highlights a need for early diagnosis and efficient management of prostate cancer.

In diagnosing prostate cancer, prostate ultrasonography has become an essential tool, but is currently mainly limited to measurement of prostate volume. In addition, because the examination cost amounts to about 150,000 Korean won at tertiary general hospitals, an economic burden may arise.

The global MRI market was valued at about 5.32 billion U.S. dollars in 2022, and is expected to grow at a compound annual growth rate of 5.9% from 2023 to 2030. In particular, an AI diagnostic market using AI algorithms is rapidly growing and is expected to reach a scale of 11.9 billion U.S. dollars by 2030.

Applicability of AI-based medical devices is being gradually expanded, and revisions to health insurance listing guidelines and recognition of interpretation reimbursement further increase stability and potential value of such technologies.

In the current medical environment in which CT and MRI imaging has become widespread, prostate ultrasonography may cause waste of health insurance finances due to redundant testing.

As of 2023, about 17,648 CT devices and 1,999 MRI devices were installed in Korea, and expansion of the supply of such medical devices and application of AI technology are leading growth of the medical market and improvement of diagnostic efficiency.

Such a technical background suggests that application of AI technology based on CT and MRI image data may be an important alternative in diagnosing prostate cancer.

Under such circumstances, The present disclosure provides an automated diagnostic support system capable of improving efficiency of prostate cancer diagnosis, solving a problem of redundant examinations, and precisely detecting a suspected cancer region using CT and MRI data.

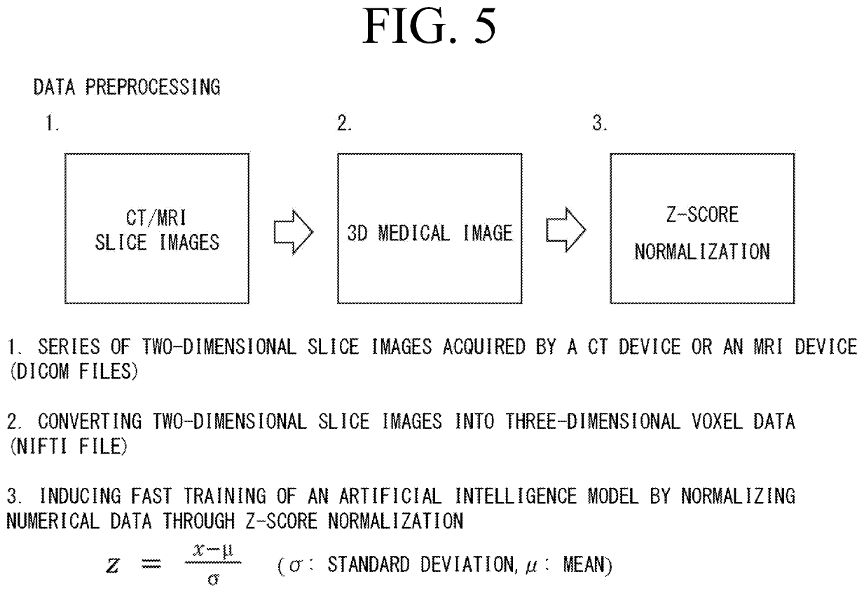

FIG. 5 illustrates an example of a preprocessing process of image data before inputting the image data to an artificial intelligence model according to various embodiments of The present disclosure.

FIG. 5 illustrates a preprocessing procedure of image data according to various embodiments of The present disclosure.

In a first step, a plurality of two-dimensional (2D) slice images including a prostate region of a patient are acquired using CT or MRI equipment. MRI and CT images are captured as multiple slices, and each slice is generally converted into and stored as a single DICOM file. These images are typically stored in a DICOM (Digital Imaging and Communications in Medicine) format and form a series of image data as a collection of slice images.

In a second step, the two-dimensional slice image data are converted into three-dimensional medical image data. The segmented DICOM files are combined into a single file to construct three-dimensional voxel data and are then converted into a NIFTI file. In this process, a concept of voxel data is used, which represents an extension of pixel units into three-dimensional space and constitutes volumetric data. The converted data are stored in a NIFTI (NIfTI-1) file format, which can be efficiently used in various medical image analysis software programs.

In a third step, numerical data are refined through Z-score normalization. Before the converted NIFTI file is input to an artificial intelligence model, Z-score normalization is applied in order to normalize the numerical data into a standard distribution. Since pixel values of medical images may vary depending on imaging equipment or acquisition environments, an artificial intelligence model may have difficulty learning the data accurately. Z-score normalization resolves such imbalance in the data and performs a process of normalizing all pixel values within a common distribution. The normalized data facilitate faster and more stable training of the artificial intelligence model.

The Z-score normalization is performed as follows. Z-score normalization subtracts an average value μ of the data from each pixel value x and divides the result by a standard deviation σ. Through this process, the data follow a normal distribution having a mean of zero and a standard deviation of one. This can be expressed by the following equation:

$$Z = ( x - μ ) / σ$$

The normalized data obtained through this process are adjusted to a consistent range across various images, thereby enabling the artificial intelligence model to learn the data more efficiently.

Through the Z-score normalization, numerical data are refined into a normal distribution while noise is reduced. In particular, imbalance in pixel values in medical images is resolved, thereby improving data quality and significantly enhancing both training speed and accuracy of the artificial intelligence model. As a result, foundational data required for detecting an entire prostate volume, subregions of the prostate, and suspected cancer regions are optimized.

The normalized data obtained through the preprocessing procedure are subsequently input to the artificial intelligence model. Based on these data, the model measures the entire prostate volume and prostate subregions and detects suspected cancer regions. Such preprocessing serves as a critical step for improving reliability of prostate cancer diagnosis by ensuring accuracy and consistency of the data.

Because three-dimensional images require a large amount of computation, inference time of an artificial intelligence model may significantly increase. Accordingly, a prostate location is first estimated from entire MRI or CT images, and only the corresponding region is cropped and used as an input to the artificial intelligence model.

For example, when a torso CT image having a resolution of 512×512 includes 200 slices, slices containing the prostate generally correspond to approximately 20 to 30 slices. In such a case, the region centered on the prostate is cropped with additional padding of approximately 10 slices before and after the prostate region, that is, approximately −10 to +10 slices, and the cropped data are converted into a three-dimensional NIFTI file.

A method of locating the prostate region includes slicing entire three-dimensional image data in frontal and lateral directions and applying a two-dimensional object detection model to precisely identify a location of the prostate. Through this process, only necessary portions of the image data can be efficiently extracted, thereby reducing computation time and optimizing performance of the artificial intelligence model.

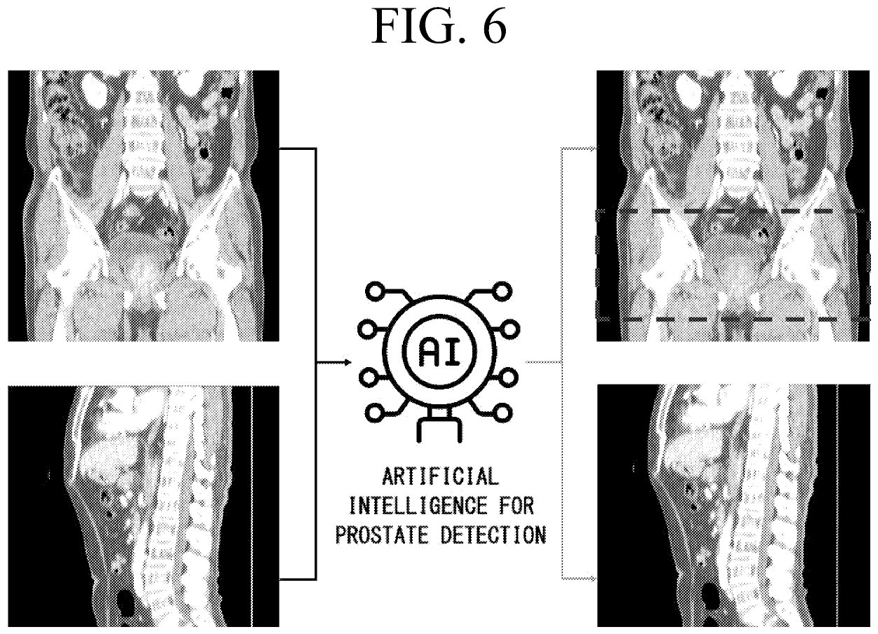

FIG. 6 illustrates an example of an artificial intelligence model for detecting prostate cancer according to various embodiments of The present disclosure.

FIG. 6 illustrates an example of a process in which an artificial intelligence model for detecting prostate cancer analyzes CT and MRI image data to detect a prostate and a suspected prostate cancer region according to various embodiments of The present disclosure. The artificial intelligence model precisely detects a prostate region from the input medical image data and visually distinguishes a suspected cancer region to present the detected result to a user.

The artificial intelligence model of The present disclosure receives, as input data, a plurality of slice images obtained using CT or MRI equipment. The anterior-posterior and lateral CT images shown on the left side represent original input data. The input data are configured in the form of two-dimensional slices, and a prostate region is detected based on the slice images.

The artificial intelligence model applies a prostate detection algorithm to detect a position and a shape of the prostate in the input image data. The artificial intelligence model illustrated in the center applies a deep learning based model, for example a convolutional neural network, that has learned characteristic features of the prostate region, thereby automatically segmenting the prostate and identifying a suspected cancer region.

As illustrated in an upper-right image of FIG. 6, the artificial intelligence model identifies a suspected cancer region after detecting the prostate region. In the illustrated example, a region displayed in green represents a suspected cancer region detected by the artificial intelligence model. The region is distinguished from surrounding normal tissue using techniques such as texture analysis or pixel value comparison.

In addition, a dashed box illustrated in FIG. 6 emphasizes a region of interest (ROI) in which the detected suspected prostate cancer region is located.

A lower-right lateral image of FIG. 6 illustrates an example in which the prostate region and the suspected cancer region are visualized from another viewing angle. The artificial intelligence model converts detection results into two-dimensional and three-dimensional visualization data and provides the visualization data to a user, thereby enabling a diagnostician to more intuitively identify the suspected cancer region.

The artificial intelligence model of The present disclosure automatically analyzes a large number of CT or MRI slice images and rapidly and accurately detects suspected prostate cancer regions. Through such analysis, diagnostic time of medical professionals may be reduced while diagnostic accuracy is improved. In addition, redundant examinations may be reduced, thereby lowering financial burdens for both patients and medical institutions. In particular, visualization results may serve as an important auxiliary tool during a prostate cancer diagnosis process.

### Artificial Intelligence Segmentation Processing

The artificial intelligence model of The present disclosure includes three models in total, including two models for measuring a prostate volume, namely a model for measuring an entire prostate region and a model for measuring prostate subregions, and one model for detecting a suspected prostate cancer region. These models use a similar network structure, but are trained with different parameters depending on a target task.

In the case of prostate subregions, the prostate is divided into a transition zone, a peripheral zone, and a central zone, and volumes of the respective zones are precisely calculated. Conventional artificial intelligence models applied a two-dimensional segmentation method in which sliced medical images were processed one by one to mask a suspected prostate lesion region and a prostate object region.

However, although prostate structures and lesion regions exhibit consistent patterns when converted into three-dimensional data, the structures appear irregular and discontinuous in two-dimensional slice images. Accordingly, artificial intelligence models have difficulty accurately learning such patterns using only two-dimensional data.

The present disclosure converts MRI and CT images into three-dimensional data and applies a three-dimensional convolutional neural network (CNN) to accurately mask a suspected lesion region and a prostate object region. Through this approach, an entire prostate volume, prostate subregions, and suspected prostate cancer regions can be detected with higher precision.

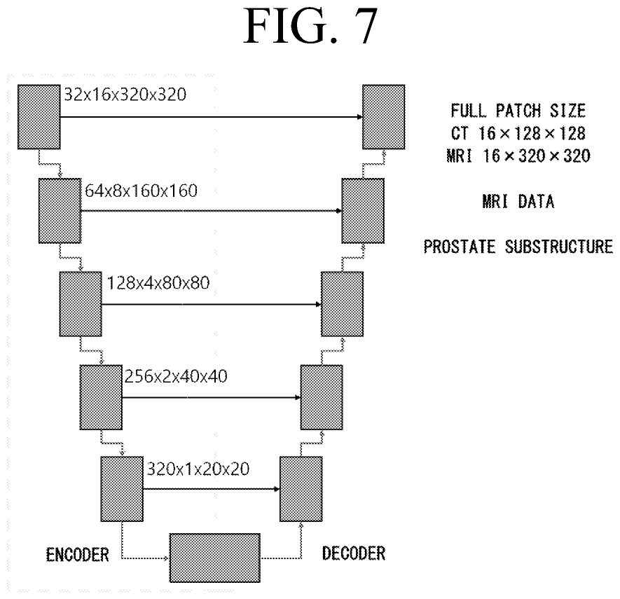

FIG. 7 illustrates an example of a 3D U-Net structure (MRI image input example) according to various embodiments of The present disclosure.

FIG. 7 illustrates an example of MRI image input using a three-dimensional U-Net (3D U-Net) architecture according to various embodiments of The present disclosure. An artificial intelligence model used in The present disclosure includes two models for measuring prostate volume, namely a model for measuring an entire prostate region and a model for measuring prostate subregions, and one model for detecting a suspected prostate cancer region. These models share the same network architecture, but are trained for different purposes by modifying detailed parameters.

For prostate subregions, volumes are calculated by dividing the prostate into a transition zone, a peripheral zone, and a central zone. Conventional two-dimensional segmentation models process medical images slice by slice, which results in lower learning efficiency due to irregular shapes of the prostate and lesion regions in individual slices. However, when the prostate is represented as three-dimensional data, more consistent patterns appear, enabling an artificial intelligence model to learn the structures more accurately.

Segmentation processing using a three-dimensional U-Net architecture is therefore applicable. In The present disclosure, MRI and CT image data are converted into three-dimensional data, and a three-dimensional convolutional neural network (CNN) is applied to mask a prostate object region and a suspected lesion region. Through this approach, a location and a shape of a lesion can be detected with higher accuracy.

An application sequence of the artificial intelligence model is as follows.

In a first step, CT and MRI image preprocessing is performed, and an entire image having a resolution of 512×512×number of slices is prepared for input into the 3D U-Net model.

In a second step, the image is divided into patch units and input into the model. A patch size of an MRI image is 16×320×320, and a patch size of a CT image is 16×128×128.

In a third step, with respect to an encoder and a decoder of the 3D U-Net architecture, the encoder extracts feature points from an input image and the decoder converts the extracted features into a masked image representing a suspected lesion region or a prostate object region. In this process, the encoder of the U-Net architecture gradually reduces image resolution while extracting features, and the decoder reconstructs the image to an original resolution using the extracted features.

In a fourth step, with respect to generation of a mask image, a mask image having the same resolution as that of the input image, namely 512×512×number of slices, is generated. The mask is a binary image composed of values 0 and 1, and a pixel having a value of “1” represents a suspected lesion region or a prostate object.

In a fifth step, with respect to prostate volume calculation, pixel information of the generated mask image is converted into a unit of cubic centimeters (cc) to calculate a prostate volume. In this process, pixel spacing information included in DICOM and NIFTI files is used to calculate an actual volume.

Finally, the calculated prostate volume and the detected suspected lesion region are output in a visualized form through a software program. Through the visualization, a user can intuitively confirm a condition of the prostate, thereby enabling more precise and efficient diagnosis of prostate cancer.

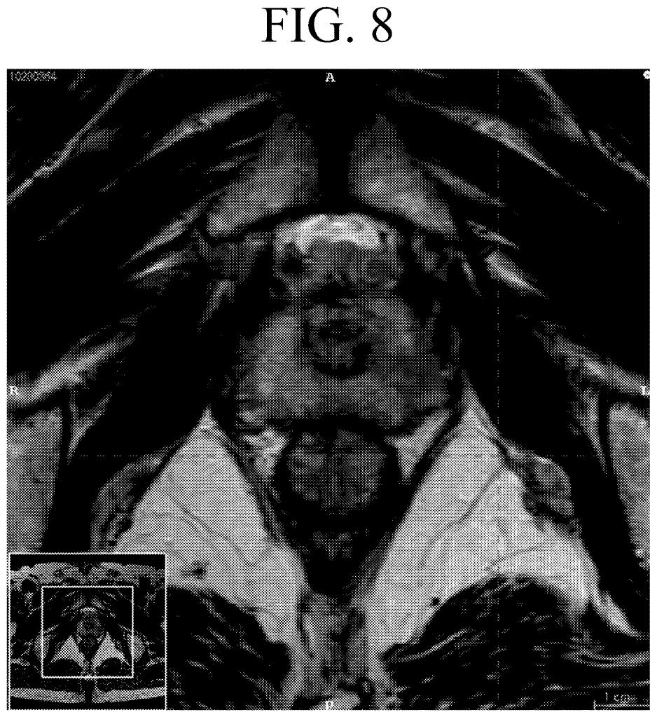

FIG. 8 illustrates an example of an output screen of an electronic device for providing determination information regarding clinically significant prostate cancer (CSPC) based on an artificial intelligence model according to various embodiments of The present disclosure.

FIG. 8 illustrates a result of automatically detecting a location, size, and boundary of a tumor in a prostate MRI image using an artificial intelligence model according to various embodiments of The present disclosure. A prostate region is displayed on the screen, and a specific portion of the prostate region is highlighted with a color, indicating a lesion detected as a suspected prostate cancer region. The artificial intelligence model analyzes various image features and automatically marks regions within the prostate that have a high probability of containing clinically significant prostate cancer (CSPC).

In the present embodiment, the artificial intelligence model generates three-dimensional voxel data from CT or MRI images and automatically segments an entire prostate region and prostate subregions. Thereafter, regions suspected of containing tumors are detected based on factors such as texture analysis, intensity contrast, and boundary characteristics, and the detection results are visually presented on the output screen. In particular, by highlighting specific lesion regions, the display assists medical personnel in quickly identifying suspicious areas at a glance.

The electronic device of The present disclosure may calculate a PI-RADS (Prostate Imaging Reporting and Data System) score by analyzing characteristics of the detected suspected prostate cancer region. The PI-RADS score is determined by comprehensively considering factors such as signal intensity of a lesion, lesion size, clarity of lesion boundaries, and contrast with surrounding tissue. Based on the analysis results, a probability of CSPC may be evaluated. In addition, information regarding a volume, location, and shape of the suspected prostate cancer region may be provided together with the PI-RADS score in order to improve accuracy of CSPC determination.

The analysis result displayed on the output screen is used to determine whether the suspected prostate cancer region corresponds to CSPC. When a detected lesion exceeds a predetermined threshold, the lesion may be classified as having a high probability of CSPC, and corresponding final determination information is provided to medical personnel. The present disclosure provides an innovative method that reduces errors that may occur in conventional manual interpretation processes and enables rapid and consistent determination of CSPC through AI-based automatic analysis.

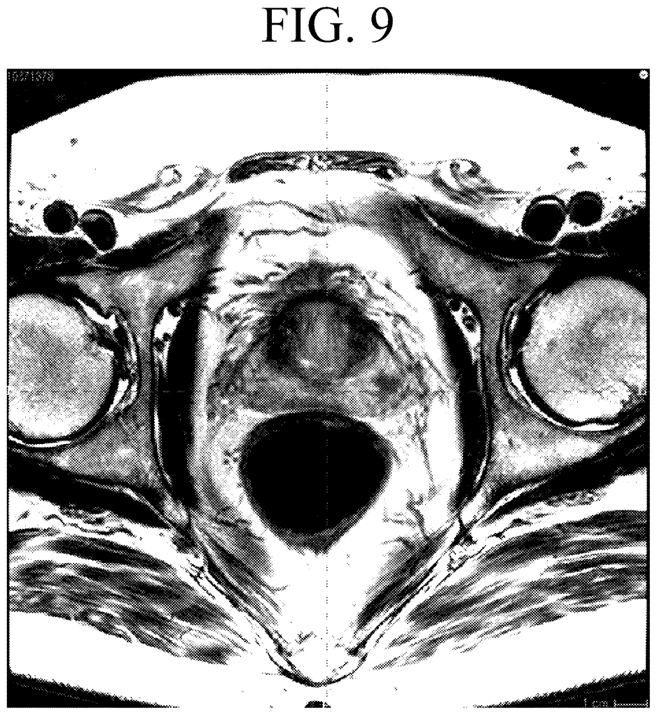

FIG. 9 illustrates another example of an output screen of an electronic device for providing determination information regarding clinically significant prostate cancer (CSPC) based on an artificial intelligence model according to various embodiments of The present disclosure.

FIG. 9 illustrates a result in which an artificial intelligence model analyzes a prostate MRI image and automatically detects a location, size, and boundary of a tumor according to various embodiments of The present disclosure. In the image, a specific region is highlighted in color, indicating a lesion detected by the AI model as a suspected prostate cancer region. The AI-based analysis method of The present disclosure automatically segments an entire prostate region and prostate subregions and subsequently detects regions suspected of containing tumors by evaluating signal intensity, contrast, and boundary clarity of lesions.

In the present embodiment, three-dimensional voxel data are generated based on medical image information, and the voxel data are input into an artificial intelligence model to detect an entire prostate region and prostate subregions. Thereafter, a PI-RADS (Prostate Imaging Reporting and Data System) score is calculated by analyzing characteristics of the detected suspected prostate cancer region, including size, shape, location, and intensity contrast. The PI-RADS score serves as an important indicator for evaluating malignancy potential of a lesion and plays a significant role in determining whether the lesion corresponds to clinically significant prostate cancer (CSPC).

The electronic device of The present disclosure determines whether CSPC is present by comprehensively considering the calculated PI-RADS score, volume information of the suspected prostate cancer region, and shape characteristics of the lesion. The AI model classifies lesions exceeding a predetermined threshold as CSPC, and assists interpretation by medical personnel using various image analysis techniques and pattern recognition methods. Through automation of image interpretation, The present disclosure provides consistent analysis results and reduces errors that may occur in conventional manual diagnosis processes.

Finally, an output screen of The present disclosure visually presents CSPC-related information so that medical personnel can intuitively confirm diagnostic results. Information regarding location, size, shape of a tumor, and a PI-RADS score are displayed together, enabling medical personnel to make more precise diagnoses and obtain key information required for establishing treatment plans. Through this approach, early detection and accurate determination of prostate cancer become possible, contributing to improvement of patient treatment processes and reduction of unnecessary biopsy procedures.

### 1. Background of the Present Disclosure

Determination of clinically significant prostate cancer (CSPC) is a critical factor in prostate cancer management. CSPC refers to prostate cancer that requires clinical treatment, and accurate identification of CSPC is essential to prevent unnecessary treatment while ensuring that appropriate treatment is provided to patients who require it. Minimizing misdiagnosis and maximizing treatment efficiency are primary goals of technologies for identifying clinically significant prostate cancer.

### 2. Objective of The present disclosure

An objective of The present disclosure is to automatically detect tumor lesions in MRI images and evaluate clinical significance of the detected lesions in order to determine clinically significant prostate cancer.

### 3. Implementation Method of the Present Disclosure

### (1) Tumor Lesion Detection:

Tumor lesions are automatically detected using deep learning techniques.

### (2) Feature Analysis:

Characteristics of lesions, including size, shape, and location, are analyzed.

### (3) Clinical Significance Evaluation:

Malignancy of lesions is evaluated using PI-RADS criteria.

### (4) Diagnostic Support System:

Risk levels of lesions are visually presented and a comprehensive analysis report is generated.

### 4. Technical Implementation Results

### Tumor Detection Using Artificial Intelligence

The present disclosure includes cases in which tumors are successfully detected and classified from prostate MRI images using artificial intelligence. By applying deep learning algorithms, locations, sizes, and shapes of tumors within the prostate can be automatically identified. This technology effectively analyzes complex image patterns and enables accurate evaluation of clinical significance of tumors.

### Performance of Clinically Significant Prostate Cancer Determination

The AI model used for evaluating clinical significance achieved an AUROC (Area Under the Receiver Operating Characteristic Curve) of 0.91 in determining whether a tumor corresponds to CSPC. This indicates that the AI model can identify clinically significant cancers with very high accuracy. Accordingly, the model assists medical professionals in making more accurate clinical decisions and contributes to early detection of prostate cancer and establishment of appropriate treatment plans. In addition, The present disclosure promotes efficient utilization of medical resources and plays an important role in improving quality of life of patients.

### Main Features of the Present Disclosure

The present disclosure relates to a method and an apparatus for automatically detecting tumor lesions in prostate MRI images and determining whether the lesions correspond to clinically significant prostate cancer (CSPC). Conventional interpretation of MRI images may vary depending on experience and subjective judgment of interpreters. The present disclosure enables more objective and precise analysis by utilizing artificial intelligence.

### Main Process of The present disclosure

### 1. Tumor Lesion Detection System

(1) Tumor lesions are automatically detected in MRI images using a deep learning model.

(2) Multi-sequence MRI data, for example T2-weighted imaging, diffusion-weighted imaging (DWI), and apparent diffusion coefficient (ADC), are integrated and analyzed to precisely map locations of lesions.

### 2. Feature Analysis System

(1) Radiomic features including size, shape, and location are automatically extracted from lesion regions.

### 3. Clinical Significance Evaluation

(1) Extracted radiomic features are standardized.

(2) Based on the standardized radiomic features, an AI algorithm determines whether the lesion corresponds to clinically significant prostate cancer.

### 4. Diagnostic Support System

(1) Risk levels of lesions are visually presented.

(2) An analysis report is automatically generated to support interpretation by medical personnel.

In a case where the embodiments of The present disclosure are implemented using hardware, Application Specific Integrated Circuits (ASICs), Digital Signal Processors (DSPs), Digital Signal Processing Devices (DSPDs), Programmable Logic Devices (PLDs), Field Programmable Gate Arrays (FPGAs), or the like, configured to perform The present disclosure, may be provided in the processor of The present disclosure.

Meanwhile, the above-described method can be written as a program executable on a computer, and can be implemented in a general-purpose digital computer that operates the program using a computer-readable medium. In addition, the structure of data used in the above-described method may be recorded on a computer-readable storage medium through various means. Program storage devices usable to describe a storage device including executable computer code to perform the various methods of The present disclosure should not be understood as including transitory subjects such as carrier waves or signals. The computer-readable storage medium includes storage media such as magnetic storage media (e.g., ROM, floppy disk, hard disk, etc.) and optical reading media (e.g., CD-ROM, DVD, etc.).

The embodiments described above are combinations of elements and features of The present disclosure in a predetermined form. Each element or feature should be considered optional unless explicitly stated otherwise. Each element or feature may be implemented in a form not combined with other elements or features. Also, it is possible to configure an embodiment of The present disclosure by combining some elements and/or features. The order of operations described in the embodiments of The present disclosure may be changed. Some configurations or features of one embodiment may be included in other embodiments, or may be replaced with corresponding configurations or features of other embodiments. It is apparent that claims that do not have an explicit citation relationship in the claims may be combined to configure an embodiment or may be included as a new claim by amendment after filing.

It will be apparent to those skilled in the art that The present disclosure may be embodied in other specific forms without departing from the technical spirit and essential characteristics of The present disclosure. Therefore, the above embodiments should be considered in all respects as illustrative and not restrictive. The scope of The present disclosure should be determined by reasonable interpretation of the appended claims and all possible changes within the equivalent scope of The present disclosure.

What is claimed is:

1. A method performed by an electronic device, the electronic device comprising an input device, an output device, a processor, and a memory, the method comprising:
acquiring, by the input device, two-dimensional medical image data related to a prostate of a patient from a magnetic resonance imaging (MRI) device or a computed tomography (CT) device;
generating, by the processor, three-dimensional voxel data of the prostate based on the two-dimensional medical image data;
acquiring, by the input device, non-image clinical data including a serum prostate-specific antigen (PSA) level of the patient and a past diagnostic history of the patient;
applying, by the processor, a feature extraction network to the three-dimensional voxel data to generate an imaging feature map, and generating a zone map identifying a peripheral zone (PZ) and a transition zone (TZ) of the prostate within the three-dimensional voxel data;
determining, by the processor, a Biomarker-Adaptive Spatial Attention Strategy based on the non-image clinical data,
wherein the strategy includes a zone sensitivity asymmetrically assigned to the peripheral zone (PZ) and the transition zone (TZ) according to the PSA level or the past diagnostic history, and a time-responsive weight according to a likelihood of lesion progression;
operating, by the processor, a multimodal cross-attention module according to the determined strategy to dynamically modulate attention weights assigned to spatial locations within the imaging feature map;
analyzing, by the processor, the modulated imaging feature map to detect an entire prostate region and one or more suspected cancer lesions, and calculating a total prostate volume and a lesion volume of the suspected cancer lesion;
calculating, by the processor, a Tumor Burden Ratio, which is a ratio of the lesion volume to the total prostate volume, and a Corrected Prostate-Specific Antigen Density (Corrected PSAD) obtained by dividing the PSA level by a non-lesion prostate volume obtained by excluding the lesion volume from the total prostate volume; and
outputting, by the output device, integrated diagnostic information including the Tumor Burden Ratio and the Corrected PSAD together with a determination result indicating whether the suspected cancer lesion corresponds to Clinically Significant Prostate Cancer (CSPC).

2. The method of claim 1,
wherein the non-image clinical data further include, in addition to the serum prostate-specific antigen (PSA) level, structured fields corresponding to the past diagnostic history, and the structured fields include at least one of information on past examination time points, summary information of past interpretation results or pathological results, information on a location of a past lesion or a zone assignment of the past lesion, and information indicating whether treatment corresponding to a prostate cancer treatment history has been performed or a type of the treatment;
wherein the feature extraction network includes a neural network configured to generate the imaging feature map by extracting voxel-level spatial features from the three-dimensional voxel data;
wherein the zone map includes, for each voxel of the three-dimensional voxel data, a zone label or a zone membership value indicating whether the voxel belongs to the peripheral zone (PZ) or the transition zone (TZ) of the prostate, and the zone membership value includes probability information or membership information represented as a continuous value;
wherein the Biomarker-Adaptive Spatial Attention Strategy includes elements configured to
(i) set a first zone sensitivity applied to spatial locations corresponding to the peripheral zone (PZ) and a second zone sensitivity applied to spatial locations corresponding to the transition zone (TZ) differently from each other, and
(ii) set a time-responsive weight such that spatial locations having a higher likelihood of lesion occurrence, expansion, or progression are relatively emphasized according to a temporal order of the past diagnostic history or a prostate cancer treatment history included in the non-image clinical data;
wherein the multimodal cross-attention module includes an attention operation configured to compute the attention weights using a clinical query vector generated by encoding the non-image clinical data as a query and the imaging feature map as keys and values;
wherein dynamically modulating the attention weights includes adjusting a distribution of the attention weights assigned to spatial locations within the imaging feature map so that the distribution is changed according to zones or according to the time-responsive weight based on a value of the clinical query vector or the zone map;
wherein the suspected cancer lesion is represented as a lesion candidate region identified based on the modulated imaging feature map and is expressed as at least one of a lesion-instance mask or a lesion candidate probability map;
wherein the Tumor Burden Ratio includes a ratio of the lesion volume of the suspected cancer lesion to the total prostate volume or a ratio of a sum of lesion volumes of suspected cancer lesions to the total prostate volume;
wherein the non-lesion prostate volume includes a volume of remaining voxels obtained by excluding voxels included in the suspected cancer lesion from voxels constituting the entire prostate region; and
wherein the Corrected Prostate-Specific Antigen Density (Corrected PSAD) includes a value obtained by dividing the PSA level by the non-lesion prostate volume.

3. The method of claim 1,
wherein the non-image clinical data include prostate-specific antigen (PSA) trend information at multiple time points, and the PSA trend information includes a time-series array of PSA values corresponding to respective time points, examination time-point metadata corresponding to the time-series array, and at least one of a change-direction feature, a change-rate feature, a change-acceleration feature, and a sudden-change event flag generated from the time-series array, and
wherein the time-responsive weight is configured as a spatially varying time-weight map in which different temporal weights are assigned to spatial locations according to at least one of the change-direction feature, the change-rate feature, the change-acceleration feature, and the sudden-change event flag.

4. The method of claim 1,
wherein the Biomarker-Adaptive Spatial Attention Strategy is configured as a strategy parameter packet including (i) a zone weight parameter set representing a relative relationship between a first zone weight corresponding to the peripheral zone (PZ) and a second zone weight corresponding to the transition zone (TZ), and (ii) a spatial distribution parameter set representing a distribution of spatial locations to which the time-responsive weight is applied,
wherein the strategy parameter packet is generated based on a combination function using, as inputs, a clinical risk context vector generated from the non-image clinical data and a zone context vector generated from the zone map.

5. The method of claim 1,
wherein the past diagnostic history includes at least one of a center coordinate of a lesion candidate, zone assignment information of the lesion candidate, and size information or size change information of the lesion candidate obtained from a past imaging examination or a past interpretation result, and
wherein the time-responsive weight is configured as a location-specific time-weight map reflecting a likelihood of lesion occurrence, expansion, or progression by being combined with a spatial prior map generated based on at least one of the center coordinate of the lesion candidate and the zone assignment information.

6. The method of claim 1,
wherein the multimodal cross-attention module generates a Key component and a Value component from the imaging feature map and generates a Query component from the clinical query vector,
wherein the zone sensitivity is configured as a zone-specific scaling tensor applied along a channel axis of the Key component or the Value component, and
wherein the zone-specific scaling tensor is masked by a zone membership channel of the zone map so that different scaling factors are applied to the peripheral zone (PZ) and the transition zone (TZ).

7. The method of claim 1,
wherein an evidence map includes a voxel-level contribution map generated based on a spatial distribution of the attention weights, and
wherein the evidence map includes a peripheral-zone evidence map corresponding to the peripheral zone (PZ) and a transition-zone evidence map corresponding to the transition zone (TZ) based on the zone map, and
wherein the integrated diagnostic information further includes display information in which the peripheral-zone evidence map and the transition-zone evidence map are overlaid on the medical image data as distinguishable layers.

8. The method of claim 1,
wherein the attention weights include zone-wise normalized attention distributions obtained by applying independent normalization operations to a peripheral-zone (PZ) domain and a transition-zone (TZ) domain separated by the zone map, and
wherein the zone-wise normalized attention distributions are configured such that normalization contributions of boundary-adjacent voxels are adjusted based on continuous zone membership values and boundary uncertainty information included in the zone map.

9. The method of claim 1,
wherein the non-lesion prostate volume is calculated using a non-lesion weight map generated based on voxel-wise lesion probability values of a probability map for the suspected cancer lesion,
wherein the non-lesion weight map is configured as a zone-adaptive non-lesion weight map in which different weighting functions are applied for respective zones according to a zone membership channel of the zone map, and
wherein an expected non-lesion volume is calculated by a weighted integration of a product of the zone-adaptive non-lesion weight map and voxel-wise volumes of the entire prostate region.

10. An electronic device comprising:
an input device; an output device; a processor; and a memory storing instructions executable by the processor,
wherein the instructions cause the processor to perform operations comprising:
acquiring, by the input device, two-dimensional medical image data related to a prostate of a patient from a magnetic resonance imaging (MRI) device or a computed tomography (CT) device;
generating, by the processor, three-dimensional voxel data of the prostate based on the two-dimensional medical image data;
acquiring, by the input device, non-image clinical data including a serum prostate-specific antigen (PSA) level of the patient and a past diagnostic history of the patient;
applying, by the processor, a feature extraction network to the three-dimensional voxel data to generate an imaging feature map, and generating a zone map identifying a peripheral zone (PZ) and a transition zone (TZ) of the prostate within the three-dimensional voxel data;
determining, by the processor, a Biomarker-Adaptive Spatial Attention Strategy based on the non-image clinical data,
wherein the strategy includes a zone sensitivity asymmetrically assigned to the peripheral zone (PZ) and the transition zone (TZ) according to the PSA level or the past diagnostic history, and a time-responsive weight according to a likelihood of lesion progression;
operating, by the processor, a multimodal cross-attention module according to the determined strategy to dynamically modulate attention weights assigned to spatial locations within the imaging feature map;
analyzing, by the processor, the modulated imaging feature map to detect an entire prostate region and one or more suspected cancer lesions, and calculating a total prostate volume and a lesion volume of the suspected cancer lesion;
calculating, by the processor, a Tumor Burden Ratio, which is a ratio of the lesion volume to the total prostate volume, and a Corrected Prostate-Specific Antigen Density (Corrected PSAD) obtained by dividing the PSA level by a non-lesion prostate volume obtained by excluding the lesion volume from the total prostate volume; and
outputting, by the output device, integrated diagnostic information including the Tumor Burden Ratio and the Corrected PSAD together with a determination result indicating whether the suspected cancer lesion corresponds to Clinically Significant Prostate Cancer (CSPC).
